SEQUENCE LISTING (6 sequences)

SEQ ID NO: 1 - DNA, 1410 bp, Artificial Sequence
  Fusion of nucleotide sequences encoding signal protein sequence from human PEDF (Serpin F1, PEDF-1) gene and human MMP1 (matrix metalloproteinase-1) gene.
  sig_peptide (1)..(57)
atgcaggccctggtgctactcctctgcattggagccctcctcgggcacagcagctgcttcccagcgactctagaaacacaagagcaagatgtggacttagtccagaaatatttggaaaaatactacaacctgaagaatgatgggcgccaggttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgaggggaaccctcgctgggagcaaacacatctgacctacaggattgaaaattacacgccagatttgccaagagcagatgtggaccatgccattgagaaagccttccaactctggagtaatgtcacacctctgacattcaccaaggtctctgagggtcaagcagacatcatgatcagctttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcagctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcgtgtgacagtaagctaacctttgatgctataactacgattcggggagaagtcatgttcttcaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaagacatttacagctcctttggcttccctagaactgtgaaacacattgatgctgctctttctgaggaaaacactggaaaaacctacttctttgttgctaacaaatactggaggtatgatgaatataaacgatctatggatccaggttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaattaa

SEQ ID NO: 2 - DNA, 57 bp, Homo sapiens
  sig_peptide (1)..(57): Nucleotide sequence of human PEDF signal peptide
atgcaggccctggtgctactcctctgcattggagccctcctcgggcacagcagctgc

SEQ ID NO: 3 - protein, 469 aa, Artificial Sequence
  Human PEDF signal peptide and human MMP1 fusion protein
  SIGNAL (1)..(19)
  PROPEP (20)..(99): Pre-pro-portion of MMP1 that gets cleaved away to allow enzymatic activation of mature polypeptide
MQALVLLLCIGALLGHSSCFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 4 - protein, 19 aa, Homo sapiens
MQALVLLLCIGALLGHSSC

SEQ ID NO: 5 - protein, 80 aa, Homo sapiens
  PEPTIDE (1)..(80): Pre-pro-MMP1; portion of sequence that gets cleaved away to allow enzymatic activation of mature polypeptide
FPATLETQEQDVDLVQKYLEKYYNLKNDGRQVEKRRNSGPVVEKLKQMQEFFGLKVTGKPDAETLKVMKQPRCGVPDVAQ

SEQ ID NO: 6 - protein, 370 aa, Homo sapiens
  mat_peptide (1)..(370): Mature (active) MMP1 protein
FVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

(19) United States
(12) Patent Application Publication
(10) Pub. No.: US 2020/0129561 A1
(43) Pub. Date: Apr. 30, 2020

(54) DELIVERY OF AUTOLOGOUS CELLS COMPRISING MATRIX METALLOPROTEINASE FOR TREATMENT OF SCLERODERMA

(71) Applicant: Intrexon Corporation, Blacksburg, VA (US)

(72) Inventors: Darby Thomas, Raleigh, NC (US); John Maslowski, Exton, PA (US); Anna Malyala, Exton, PA (US)

(73) Assignee: Intrexon Corporation, Blacksburg, VA (US)

(21) Appl. No.: 16/606,293

(22) PCT Filed: Apr. 20, 2018
(86) PCT No.: PCT/US2018/028551

§ 371 Date: Oct. 18, 2019

Related U.S. Application Data
(60) Provisional application No. 62/488,207, filed on Apr. 21, 2017.
(60) Provisional application No. 62/512,382, filed on May 30, 2017.

(51) Int. Cl. A61K 35/33; C12N 15/86; A61K 38/48; A61K 31/15; A61K 9/00; A61P 19/04

(52) U.S. Cl. CPC A61K 35/33; C12N 15/86; A61P 19/04; A61K 31/15; A61K 9/0021; A61K 38/4886

(57) ABSTRACT

The present invention relates to a method for the treatment of scleroderma through the delivery of matrix metalloproteinase (MMP) to a patient in need thereof, preferably under the control of a gene switch. In this manner, the use of a ligand activator to activate or deactivate the expression of MMP controls the gene switch. In another embodiment, the invention is directed to the delivery of autologous genetically modified cells transfected/transduced with a polynucleotide encoding MMP under the control of a gene switch activatable through the use of an activator ligand for the treatment or scleroderma.

### REFERENCE TO SEQUENCE LISTING

The instant application contains a Sequence Listing which has been submitted electronically in ASCII format and is hereby incorporated by reference in its entirety. Said ASCII copy, created on Apr. 20, 2018, is named “INX00372WO_Sequence_Listing_20180420.txt” and is 11,691 bytes in size (12,288 bytes size on disk).

### FIELD OF THE INVENTION

The present invention relates to methods and compositions for the treatment of sclerotic conditions through delivery of polynucleotides encoding matrix metalloproteinase (MMP) or a collagen-degrading fragment thereof to a patient in need thereof. In a further embodiment, the invention is directed to the delivery of polynucleotides encoding MMP in a vector, which is conditionally expressed through the use of a gene switch expression system, to a cell isolated from a patient suffering from sclerotic conditions. The cell is preferably isolated from the patient, transfected with a polynucleotide encoding MMP, cultured in vitro or ex vivo, and is subsequently administered to the patient. Furthermore, a ligand activator is administered to the patient to activate, or ceased from administration to deactivate, expression of MMP. In another embodiment, the invention is directed to the constructs used to deliver the MMP or fragment thereof.

### BACKGROUND OF THE INVENTION

Localized scleroderma is an autoimmune inflammatory sclerosing disorder of cutaneous induration that may cause permanent functional disability and disfigurement. The term “morphea” is synonymous with localized scleroderma. Localized scleroderma has several subtypes including linear scleroderma, circumscribed morphea, generalized morphea, pansclerotic morphea, and mixed morphea. Localized scleroderma is a rare fibrosing disorder of the skin and underlying tissues without vascular or internal organ involvement and encompasses several subtypes classified by depth and pattern of the lesion(s). The traditional classification system (Peterson et al, 1997) has recently been modified by a consensus of experts to provide more clinically applicable classifications. The modified classification system is referred to as the “Padua criteria” (Laxer & Zulian, 2006). The underlying pathogenesis of localized scleroderma is likely multifactorial, involving genetic factors and environmental exposures, culminating in small vessel damage, the release of profibrotic cytokines, and disruption of the balance between collagen synthesis and destruction. Overproduction and accumulation of collagen is a hallmark of the disease. Linear subtype, the most common subtype in children with localized scleroderma (Fett & Werth, 2011), is characterized by linear plaques involving the dermis, subcutis, and sometimes, the underlying muscle, tendons and bone (Laxer & Zulian, 2006). Linear scleroderma is usually limited to the skin and subcutaneous tissue such as fatty tissue, muscle, and sometimes bone beneath cutaneous lesions. Localized scleroderma is usually a self-limiting problem in which the linear areas of skin thickening may extend to underlying tissue and muscle in children, which may impair growth in affected leg or arms. Indeed, the most common sites involved are the legs, followed by arms, frontal head and trunk. Lesions of the limbs may cause atrophy of soft tissue including muscle, limb length discrepancy due to impaired growth, and joint contractures. Lesions across joints impair motion and may be permanent.

The incidence and prevalence of localized scleroderma is poorly described. The incidence of localized scleroderma has been estimated as approximately 0.4 to 2.7 per 100,000 (Peterson et al. 1997) (Fett & Werth, 2011), (Kelsey & Torok, 2013). Systemic and localized scleroderma are clinically distinct diseases (Lipsker et al., 2015). According to the Scleroderma Foundation and the NIH, the US prevalence of scleroderma (systemic and localized forms) is approximately 300,000 and ⅔ of those individuals (or approximately 200,000) are estimated to have localized scleroderma (GARD, 2012) (Foundation, 2015).

Presently available information indicates, there are no curative therapies and there are no therapies specifically indicated for the treatment of localized scleroderma. Current treatment is aimed at controlling the signs and symptoms and slowing the spread of the disease. Methotrexate, corticosteroids and mycophenolate mofetil provide benefit to many patients early in the disease process, but often fail to provide long-term efficacy, particularly in lesions that have become sclerotic. As such, localized scleroderma can be considered to have two components in terms of disease progression: 1) an active (inflammatory) phase and 2) a damage (sclerotic) phase. Current treatments such as methotrexate address the active phase; however, to date no therapies are effective once a lesion is in the damage phase.

Given the rarity of localized scleroderma, only few evidence-based therapeutic treatment options exist. In general, treatment options can be divided into topical and systemic therapy, and ultraviolet (UV) phototherapy.

Although studies provide evidence that methotrexate is an effective treatment, low doses must be administered for years to suppress the condition until spontaneous improvement in disease activity occurs, but does not cure the condition (Christen-Zaech et al., 2008). Stabilization is obtained after a mean disease duration of 5.4 years. Patients sometimes experience long stretches of disease quiescence followed by reactivation; 31% of patients have reported active disease after 10 years. Most patients have aesthetic sequelae, and 38% have functional limitations. Although the combination of methotrexate and systemic corticosteroids is effective in the early stages of the disease, it does not prevent long-standing active disease or relapse in the long term (Piram et al., 2013).

UVA1 phototherapy can be efficacious; however, the treatment can be burdensome (2-3×/week for 30-40 sessions) and the recurrence rate after treatment is 46% (Piram et al., 2013). While most investigators agree that UVA1 is an effective treatment for localized scleroderma, there is a lack of consensus on the dosing regimen or frequency and total exposure.

Accordingly, new and improved therapies are needed for the treatment of scleroderma.

### BRIEF DESCRIPTION OF THE DRAWINGS

A more complete understanding of the present invention may be obtained by reference to the accompanying drawings, when considered in conjunction with the subsequent detailed description. The embodiments illustrated in the drawings are intended only to exemplify the invention and should not be construed as limiting the invention to the illustrated embodiments.

FIG. 1 depicts a gene switch system that may be used in the present invention.

FIG. 2 depicts a construct map for the lentiviral vector comprising an ecdysone receptor-based ligand-inducible gene switch and the gene encoding the MMP1 protein (“LV-RTS-MMP1”). The elements and functions of the construct are set forth in Table 1.

FIG. 3 provides a schematic of a lentivirus transduction process within the scope of the present invention.

FIG. 4 provides a graph showing the transduction of normal human dermal fibroblasts with varying dilutions of LV-RTS-MMP1 with and without veledimex as well as the average copy numbers of such transductions.

FIGS. 5A-5B provide a graphical representation showing reduced dermal thickness (FIG. 5A) and reduced sub-dermal muscle thickness (FIG. 5B) in bleomycin models of scleroderma. Group 1 corresponds to a bleomycin mouse model injected with human dermal fibroblasts (HDF) transduced with LV-RTS-MMP1 (“HDF-RTS-MMP1”) cells in which a mock excipient is orally administered. Group 2 corresponds to a bleomycin mouse model injected with HDF-RTS-MMP1 cells and orally administered veledimex. Group 3 corresponds to a bleomycin mouse model injected with non-genetically modified human dermal fibroblasts (non-GM HDFs) in which veledimex is orally administered. Group 4 corresponds to the control mouse injected with saline (not bleomycin), not injected with any types of cells, and administered oral veledimex. Error bars represent standard deviations

FIG. 6 provides a graph of the serum levels of MMP1 in mice intradermally injected with HDF-RTS-MMP1 cells. Serum was collected from each mouse on study days 28, 33 and 39 (days (−)1 [i.e., pre-bleed], 4, and 10 post-injection of fibroblasts). Serum was diluted 1:2 and assayed for MMP1 expression using a sensitive MMP1 ELISA (LLOD=22 pg/mL). Error bars represent standard deviations.

FIG. 7 provides representative in vivo pharmacology images of histological sections stained with hematoxylin and eosin (H&E) from a mouse for each of Groups 1-4

FIG. 8 shows overview of an exemplary contemplated treatment schedule.

### SUMMARY OF THE INVENTION

The present invention relates to a method for the treatment of scleroderma through the delivery of matrix metalloproteinase (MMP) to a patient in need thereof, preferably under the control of an inducible gene switch. In such manner, for example, the use of a ligand activator to activate or deactivate expression of MMP controls the gene switch (i.e., via administration or cessation of administration of a gene expression activating ligand, respectively). In another embodiment, the invention is directed to the delivery of autologous genetically modified cells transfected or transduced with a polynucleotide encoding MMP under the control of a gene switch expression through the use of an activator ligand for the treatment of scleroderma.

Embodiments of the invention include, without limitation:

A method of treating a sclerotic condition comprising administering cells that have been transfected with an expression vector comprising a polynucleotide encoding matrix metalloproteinase (MMP) protein or a collagen-degrading fragment thereof to a patient in need thereof. A method of treating a sclerotic condition, further comprising use of transfected autologous cells isolated from a patient suffering from scleroderma prior to transfection. A method of treating a sclerotic condition, further comprising use of transfected cells which are cultured ex vivo. A method of treating a sclerotic condition, further comprising use of fibroblast cells. A method of treating a sclerotic condition, further comprising use and expression of a polynucleotide encoding MMP, or a collagen-degrading fragment thereof, operably linked to a gene switch expression system. A method of treating a sclerotic condition, wherein the gene switch expression system is activated (i.e., induced or turned “on”) in the presence of an activator ligand and deactivated (i.e., reduced or turned “off”) in the absence of the activator ligand. A method of treating a sclerotic condition, wherein a gene switch expression system comprises an inducible promoter operably linked to a ligand-inducible transcription factor, which is activated when bound to the activator ligand. A method of treating a sclerotic condition, wherein the gene switch expression system further comprises a co-activation partner. A method of treating a sclerotic condition, wherein the expression vector is a viral vector. A method of treating a sclerotic condition, wherein the viral vector is derived from a virus selected from lentivirus, adenovirus, and adeno-associated virus. A method of treating a sclerotic condition, wherein the viral vector is a lentiviral vector. A method of treating a sclerotic condition, wherein said lentiviral vector is INXN-2005. A method of treating a sclerotic condition, wherein the gene expression system activator ligand is a non-steroidal compound. A method of treating a sclerotic condition, wherein the activator ligand is a non-steroidal diacylhydrazine compound. A method of treating a sclerotic condition, wherein the activator ligand is veledimex. A method of treating a sclerotic condition, wherein the cells are transfected by an expression vector comprising the polynucleotide encoding MMP or a collagen-degrading fragment thereof operably linked to a gene switch system. A method of treating a sclerotic condition, wherein the transfected cells are administered to a patient in need thereof by injection. A method of treating a sclerotic condition, wherein administration is by intradermal injection. A method of treating a sclerotic condition, wherein an activator ligand, such as but not limited to, veledimex, is administered to the patient following injection of the transfected cells. A method of treating a sclerotic condition, wherein administration of an activator ligand, such as but not limited to, veledimex, activates the gene switch to induce expression of the polynucleotide encoding the MMP or collage-degrading fragment thereof in the patient. A method of treating a sclerotic condition, wherein an activator ligand, such as but not limited to, veledimex, is delivered for at least five days after administration of the transfected cells. A method of treating a sclerotic condition, wherein an activator ligand, such as but not limited to, veledimex, is delivered daily or at other intervals for 7 days or more, 10 days or more, 14 days or more, 21 days or more, 28 days or more, 30 days or more, 60 days or more, 90 days or more, or up to 100 days or more after the administration of the transfected cells. A method of treating a sclerotic condition, wherein the sclerotic condition is localized scleroderma. A method of treating a sclerotic condition, wherein the localized scleroderma is selected from linear scleroderma, circumscribed morphea, generalized morphea, pansclerotic morphea, and mixed morphea.

A method of treating a sclerotic condition comprising administering to a patient in need thereof an intradermal injection comprising autologous cells transduced with a polynucleotide encoding matrix metalloproteinase (MMP) protein or a collagen-degrading fragment thereof operably linked to a gene switch in combination with an activator ligand that induces said gene switch. A method of treating a sclerotic condition, wherein the activator ligand of the gene switch is veledimex. A method of treating a sclerotic condition, wherein veledimex is withheld from the patient to deactivate the gene switch. A method of treating a sclerotic condition, wherein the sclerotic condition is localized scleroderma. A method of treating a sclerotic condition, wherein the localized scleroderma is selected from linear scleroderma, circumscribed morphea, generalized morphea, pansclerotic morphea, and mixed morphea.

A lentiviral vector comprising a polynucleotide encoding matrix metalloproteinase (MMP), or a collagen-degrading fragment thereof, operably linked to a gene switch system.

A lentiviral vector comprising a polynucleotide encoding matrix metalloproteinase (MMP), or a collagen-degrading fragment thereof, wherein the gene switch system comprises an inducible promoter operably linked to a ligand-inducible transcription factor, which is activated when bound to the activator ligand. A lentiviral vector, wherein the gene switch system is activated in the presence of an activator ligand and deactivated in the absence of the activator ligand. A lentiviral vector comprising the sequence of SEQ ID NO:1. A pharmaceutical composition comprising a fibroblast obtained from a patient suffering from scleroderma transduced with a lentiviral vector designated INXN-2005 comprising a nucleotide sequence as shown in SEQ ID NO:1. A pharmaceutical composition comprising a fibroblast obtained from a patient suffering from scleroderma transduced with a lentiviral vector designated INXN-2005. A cell transduced in vitro or ex vivo with a lentiviral vector comprising a polynucleotide encoding matrix metalloproteinase (MMP), or a collagen-degrading fragment thereof. A cell transduced in vitro or ex vivo with a lentiviral vector comprising the sequence of SEQ ID NO:1. An autologous genetically modified fibroblast from a patient suffering from scleroderma comprising a functional MMP gene and expresses matrix metalloproteinase-1.

An autologous genetically modified fibroblast from a patient suffering from sclerotic disease comprising a functional MMP gene and expresses matrix metalloproteinase.

### DETAILED DESCRIPTION OF THE INVENTION

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference. In the event that any statements, conclusions, hypotheses, data or other information presented in any of the cited in references conflicts with or contradicts the present disclosure, the present disclosure shall overrule and supersede the conflicting or contradictory portion of the cited reference.

In order that the present invention may be more readily understood, certain terms are herein defined. Additional definitions are set forth throughout the detailed description.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects. Typically, the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the invention, and vice versa. Furthermore, compositions of the invention can be used to achieve methods of the invention.

The terms, “nucleic acid,” “nucleic acid molecule,” “oligonucleotide,” and “polynucleotide” are used interchangeably to refer to the either RNA or DNA, along with synthetic nucleotide analogs or other molecules that may be present in the sequence and that do not prevent hybridization of the polynucleotide with a second molecule having a complementary sequence. These molecules can be either single stranded or double stranded.

Nucleic acids, nucleic acid sequences, oligonucleotides and polynucleotides are “homologous” when they are derived, naturally or artificially, from a common ancestral nucleic acid or nucleic acid sequence. Proteins and/or protein sequences are homologous when their encoding DNAs are derived, naturally or artificially, from a common ancestral nucleic acid or nucleic acid sequence. The homologous molecules can be termed homologs. For example, any naturally occurring proteins, as described herein, can be modified by any available mutagenesis method. When expressed, this mutagenized nucleic acid encodes a polypeptide that is homologous to the protein encoded by the original nucleic acid. Homology is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence identity percentages (e.g., BLASTP and BLASTN using default parameters) are described herein and are generally available.

The terms “identical” or “sequence identity” in the context of two nucleic acid sequences or amino acid sequences of polypeptides refers to the residues in the two sequences which are the same when aligned for maximum correspondence over a specified comparison window. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman (1981) Adv. Appl. Math. 2:482; by the alignment algorithm of Needleman and Wunsch (1970) J. Mol. Biol. 48:443; by the search for similarity method of Pearson and Lipman (1988) Proc. Nat. Acad. Sci U.S.A. 85:2444; by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp (1988) Gene 73:237-244 and Higgins and Sharp (1989) CABIOS 5:151-153; Corpet et al. (1988) Nucleic Acids Res. 16:10881-10890; Huang et al (1992) Computer Applications in the Biosciences 8:155-165; and Pearson et al. (1994) Methods in Molecular Biology 24:307-331. Alignment is also often performed by inspection and manual alignment.

In one class of embodiments, the polypeptides (such as fragments of an MMP, e.g., MMP-1 protein) herein are at least 70%, generally at least 75%, optionally at least 80%, 85%, 90%, 98% or 99% or more identical to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 98%, 99% or more identical to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

The term “substantially identical” as applied to nucleic acid or amino acid sequences means that a nucleic acid or amino acid sequence comprises a sequence that has at least 90% sequence identity or more, preferably at least 95%, more preferably at least 98% and most preferably at least 99%, compared to a reference sequence using the programs described above (preferably BLAST) using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

A “functional variant” of a protein disclosed herein can, for example, comprise the amino acid sequence of the reference protein (such as an MMP, e.g., MMP-1) with at least or about 1, 2, 3, 4, 5, 6, 7, 8, 9, 10, 11, 12, 13, 14, 15, 16, 17, 18, 19, or 20 conservative amino acid substitutions. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz, G. E. and Schirmer, R. H., supra). Examples of conservative mutations include amino acid substitutions of amino acids within the sub-groups above, for example, lysine for arginine and vice versa such that a positive charge may be maintained; glutamic acid for aspartic acid and vice versa such that a negative charge may be maintained; serine for threonine such that a free —OH can be maintained; and glutamine for asparagine such that a free —NH₂ can be maintained.

Alternatively or additionally, the functional variants can comprise the amino acid sequence of the reference protein with at least one non-conservative amino acid substitution. “Non-conservative mutations” involve amino acid substitutions between different groups, for example, lysine for tryptophan, or phenylalanine for serine, etc. In this case, it is preferable for the non-conservative amino acid substitution to not interfere with, or inhibit the biological activity of, the functional variant. The non-conservative amino acid substitution may enhance the biological activity of the functional variant, such that the biological activity of the functional variant is increased as compared to the reference sequence.

A “collagen-degrading” fragment of MMP is a fragment of MMP, which is capable of cleaving or degrading collagens of type I, II, and/or III. This function serves to prevent or inhibit extracellular matrix accumulation and/or provides an anti-fibrosis effect.

The “gene switch system” refers to the conditional gene expression system that allows for the expression of the reference protein, such as MMP or fragment thereof, to be turned on and off. The gene switch system of the present invention refers to a system comprising a polynucleotide sequence comprising at least one inducible promoter, which is linked to the expression of a therapeutic protein (e.g., MMP-1) or fragment thereof operably linked thereto, a ligand-inducible transcription factor, and a co-activation partner for the ligand-inducible transcription factor. In one aspect of the present invention, the “inducible promoter” is operably linked to the polynucleotide encoding the MMP or fragment thereof. The gene switch system includes the use of an “activator ligand” which, when complexed with the “ligand-inducible transcription factor,” triggers the inducible promoter to initiate transcription of the therapeutic protein or fragment thereof. Accordingly, the present invention further relates to an “activation complex” comprising the ligand-inducible transcription factor and the activator ligand to trigger the expression of MMP or fragment thereof in a patient. In that case, the activation complex would comprise the ligand-inducible transcription factor along with the co-activation partner complexed with the activator ligand in order to trigger the inducible promoter. The gene switch system is “activated” or “turned on” in the presence of the activator ligand and is “deactivated” or “turned off” in the absence of the activator ligand. Accordingly, the gene switch can be turned on and off as needed by the presence or absence of the activator ligand. In certain embodiments, a preferred gene switch expression system utilized in the present invention is an ecdysone receptor-based ligand-inducible gene switch, such as described, for example, in PCT/US2002/005090 (filed Feb. 20, 2002) and U.S. Pat. No. 8,715,959 (issued May 6, 2014) and/or in PCT/US2008/011270 and U.S. Pat. No. 9,402,919, which are herein incorporated by reference.

The term “patient” or “subject” refers to mammals, including humans and animals.

The term “treating” or “treatment” refers to reducing or alleviating the symptoms and/or preventing relapses and/or the progression of sclerotic conditions. For example, treatment of scleroderma is directed to the degradation of collagen or inhibition or prevention of extracellular matrix or collagen formation, which plays a significant role in the sclerotic conditions. Treatment may involve binding, blocking, inhibiting or neutralizing ECM production or collagen formation or the reducing, preventing or inhibiting of ECM production or collagen formation attributed to scleroderma. Indications that may be treated with the methods and compositions described herein include but are not limited to: 1) Systemic Scleroderma (SSc) (specifically, scerodactyly and internal organ fibrosis); 2) skin fibrosis including: a) limited cutaneous SSc (ISSc) and b) diffuse cutaneous SSc (dSSc); 3) systemic sceroderma with interstitial lung disease (ILD) (SSc-ILD); 4) Edematous fibrosclerotic panniculopathy (cellulite); 5) Adhesive capsulitis (frozen shoulder syndrome); 6) Raynaud's phenomenon (RP); 7) psoriasis; 8) liver fibrosis (including nonalcoholic steatohepatitis); 9) kidney fibrosis (including, focal segmental glomerulosclerosis); 10) cardiac fibrosis; 11) rheumatoid arthritis; 12) Crohn's disease; 13) ulcerative colitis; 14) myelofibrosis; 15) systemic lupus erythematosus (SLE); 16) skeletal muscle fibrosis (following acute injury or due to chronic neurodegenerative muscular diseases); 17) congenital fibrosis of the extraocular muscle (CFEOM1, CFEOM2, CFEOM3 and Tukel syndrome); 18) chronic graft versus host disease; 19) hypertrophic scars; 20) idiopathic pulmonary fibrosis; 21) en coup de sabre; 22) dupuytren's contracture; 23) peyronie's disease; 24) hypertropic scars; 25) scleroderma associated hand dysfunction; 26) radiation fibrosis syndrome; and, 27) other sclerotic conditions.

The term “autologous cells” refers to cells derived from the same individual or involving one individual as both donor and recipient. In accordance with the present invention, autologous cells are first harvested from a patient suffering from sclerodera. These cells are genetically modified in accordance with the present invention and subsequently reintroduced back into the same patient to the treat the sclerotic condition.

The term, “transfection” refers to the delivery of a gene(s) into mammalian cells. The insertion of such genetic material enables the expression, or production, of proteins using the cells own machinery. In accordance with the present invention, transfection also may refer to a cell that is transduced via the use of a viral vector or transfected via the use of chemical or electrical delivery of polynucleotides.

The term “transduction” refers to the delivery of a gene(s) using a viral or retroviral vector by means of viral infection rather than by transfection.

The term “adeno-associated viral vector” refers to a member of the Parvovirus family, and is a small non-enveloped, icosahedral virus with a single-stranded linear DNA genome. The adeno-associated virus genomes contains inverted terminal repeats (ITRs), which allow for the integration of a transduced gene into the host cell genome.

The term “transducing vector” refers to the infectious viral or viral-like vectors such as for example, herpes viruses, baculoviruses, vaccinia virus, adenoviral, lentiviral or adeno-associated viral vector particles formed from the co-transfection of a packaging cell line with the expression/transfer plasmid vector comprising the WP gene or gene encoding a collagen-degrading MMP fragment thereof, a packaging vector(s), and an envelope vector. The transducing vector is harvested from the supernatant of the producer cell culture after transfection. Suitable packaging cell lines are known in the art and include, for example, the 293T cell line.

The term “transgene” refers to any heterologous gene (i.e., any non-naturally occurring or not-normally present gene) introduced into a cell or genome.

The term “lentiviral vector” refers to a vector containing structural and functional genetic elements outside the LTRs that are primarily derived from a lentivirus.

Matrix metalloproteinase or “MMP” as used herein are calcium-dependent zinc-containing endopeptidases including adamalysins, serralysins, and astacins. The MMPs belong to a larger family of proteases known as the metzincin superfamily. The collagenase MMPs are capable of degrading triple-helical fibrillar collagens into distinctive ¾ and ¼ fragments. Preferably MMPs include but are not limited to MMP-1, MMP-2, MMP-4, MMP-7, MMP-8, MMP-9, MMP-11, MMP-13, and MMP-14.

Collectively, these enzymes are capable of degrading all kinds of extracellular matrix proteins, but also can process a number of bioactive molecules. They are known to be involved in the cleavage of cell surface receptors, the release of apoptotic ligands (such as the FAS ligand), and chemokine/cytokine inactivation. MMPs are also thought to play a major role in cell behaviors such as cell proliferation, migration (adhesion/dispersion), differentiation, angiogenesis, apoptosis, and host defense.

In particular, MMP1 digests the main constitutive proteins in fibrous scar tissue, native fibrillary collagens type I and III, while sparing collagen type IV, which is a component of the basement membrane. MMP1 may also play important roles in ECM remodeling and cell signaling by acting on the cell surface, matrix and non-matrix substrates, such and IGF binding proteins, L-selectin, and TNFα (Pardo & Selman, 2005). MMP1 is expressed as a zymogen (its “pro” form) where step-wise proteolytic cleavage is required for activation. A conserved cysteine within the pro-domain is required for maintaining MMP1 in the inactive state through binding to the zinc ion within the catalytic site (known as the “cysteine switch”). Specifically, MMP1 cleaves collagens of types I, II, and III at one site in the triple helical domain at about three-quarters of the length of the molecule from the N-terminus.

The present invention relates to delivery of cells transfected or transduced with the polynucleotide encoding MMP or a collagen-degrading fragment thereof to a patient suffering from a sclerotic condition. In an embodiment of the invention, the polynucleotide encoding MMP or a fragment thereof is delivered in a viral vector. Preferably the viral vector is a lentiviral vector. In another embodiment, the viral vector, such as a lentiviral vector, includes a gene switch system, which allows for the conditional expression of MMP or a collagen-degrading fragment thereof in the presence of an activator ligand. Where a gene switch system is used, an activator ligand is administered either prior to, concurrently or following the administration of the MMP vector. The activator ligand may be periodically administered to the patient (over a continuous period of time) or withheld from administration in a manner sufficient to allow for turning on or off gene switch and, in turn, the expression of MMP or a collagen-degrading fragment thereof. In another aspect of the invention, cells harvested from the a sclerotic condition patient is transduced with a lentiviral vector comprising a polynucleotide sequence encoding MMP or a fragment thereof and a gene switch system operably linked to the polynucleotide sequence and the transduced cells are cultured and administered to the same sclerotic conditions patient. In the case where a gene switch system is used, the ligand activator is administered to activate the gene switch or withheld to deactivate the gene switch.

Generally as referred to herein, “MMP or collagen-degrading fragment thereof” is (i) MMP; (ii) a functional variant of MMP; (iii) a protein substantially identical to MMP; (iv) a collagen-degrading fragment of MMP; or (v) a biologically active fragment of (i), (ii), (iii) or (iv).

In accordance with the present invention, the nucleotide sequence of a vector encoding MMP1 comprises a nucleotide sequence at least 80%, at least 90%, at least 95%, at least 97%, at least 99%, or 100% identical to the nucleotide sequence of SEQ ID NO:1. In SEQ ID NO:1, the sequence for the MMP1 cDNA was derived from the consensus sequence of human pro-MMP1 with a replacement of the native MMP1 signal peptide with the signal peptide sequence of human Pigment Epithelium-Derived Factor (PEDF)(SEQ ID NO:2) to provide more efficient secretion of MMP1 from fibroblasts. The cDNA (1410 bp) was generated and cloned into a standard expression plasmid for initial analysis. Notably, other signal peptide sequences may also be used instead of, in place of, the PEDF signal peptide. The MMP1 cDNA for that vector was then engineered to remove potential splice sites and cloned into a pFUGW SIN-LV backbone (see e.g., Miyoshi, et al., 72(10):8150-8157 (October 1998) and WO2017161180A1 (PCT PCT/US2017/022800)) adjacent to an inducible gene switch expression cassette (i.e., an ecdysone receptor-based gene switch), for MMP1 expression control using veledimex (activator ligand), to produce the LV-RTS-MMP1 vector (also referred to as INXN-2005).

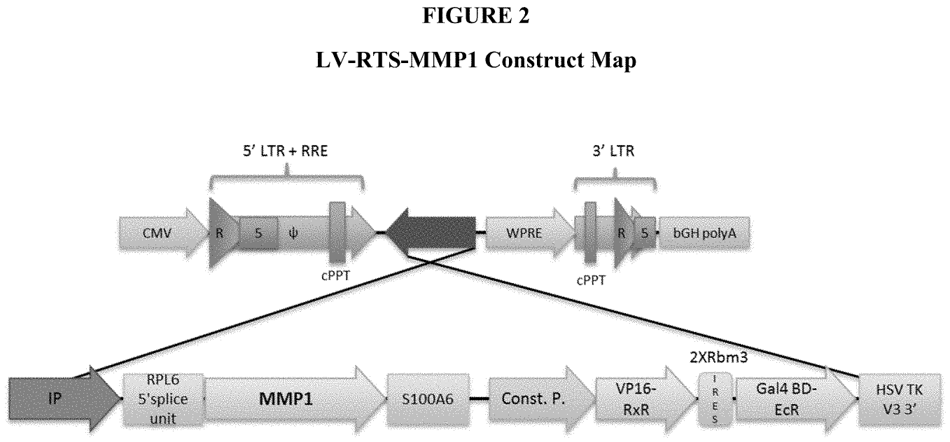

The amino acid sequence of MMP1 as expressed by the LV-RTS-MMP1 vector has the sequence of SEQ ID NO:3, in which the sequence of SEQ ID NO:4 corresponds to human PEDF signal peptide, in which the sequence of SEQ ID NO:5 corresponds to the pre-pro-MMP1 sequence, which is cleaved upon activation to form the mature (enzymatically active) MMP1 protein which comprises the amino acid sequence of SEQ ID NO:6. FIG. 2 provides a graphical representation of the LV-RTS-MMP1.

It is noted that the LV-RTS-MMP1 vector is also known as the vector “INXN-2005”. Vectors substantially identical and/or homologous to the LV-RTS-MMP1 vector are envisaged by the present invention. The INXN-2005 vector utilizes a lentiviral vector (LV) comprising a gene switch system. The LV is a replication incompetent, Vesicular Stomatitis Virus-G (VSV-G) pseudotyped, self-inactivating (3^{rd} generation) lentivirus (SIN-LV). In particular, INXN-2005 (LV-RTS-MMP1) utilizes an lentiviral platform in combination with an ecdysone receptor-based ligand-inducible gene switch expression system (such as described in in PCT/US2002/005090 and U.S. Pat. No. 8,715,959 and/or in PCT/US2008/011270 and U.S. Pat. No. 9,402,919) to conditionally express a the MMP-1 protein. The lentivirus backbone contains the minimal essential elements needed for transcription of the recombinant LV genome to be packaged into virus. Encoded within the LV backbone are the elements needed for the veledimex ligand-inducible gene switch controlled expression of MMP1. In some embodiments, the starting material to construct a transducing lentiviral vector of the present invention is selected from the lentiviral expression plasmid vectors, pSMPUW (Cell Biolabs, Inc., San Diego, Calif.) and pFUGW (Addgene, Cambridge, Mass.).

The elements of the LV-RTS-MMP1 and their function are listed below in Table 1.

TABLE 1
Description of Components of LV-RTS-MMP1

| | | |
|---|---|---|
| Abbreviation | Element | Purpose |

Lentivirus backbone

| | | |
|---|---|---|
| CMV | Cytomegalovirus immediate | Drives transcription to generate LV |
| | early promoter | mRNA genomes |
| 5′LTR: | 5′ Long Terminal Repeat region: | Facilitates LV mRNA genome |
| R | HIV Repeat region | transcription, gene expression, |
| 5 | HIV 5′ untranslated region | packaging of into viral particles, and |
| Ψ | packaging signal | integration into the host genome |
| cPPT | Central Polypurine Tract | |
| RRE | Rev-responsive Element | Facilitates transport of LV mRNA to |
| | | nucleus |
| WPRE | Woodchuck Hepatitis Virus | Increases gene expression and viral |
| | Post-transcriptional | titer |
| | regulatory element | |
| 3′LTR | 3′ Long Terminal Repeat | Contains truncated (SIN) U3 region, R |
| | region | and U5 regions of the HIV1 genome. |
| | | The truncation of the U3 removes the |
| | | promoter and enhancer elements |
| | | naturally occurring in the U3 region of |
| | | HIV1 3′LTR thus generating a self- |
| | | inactivating LV |
| bGH polyA | polyA tail sequence derived | Provides to the LV mRNA genome |
| | from the bovine growth | transcript |
| | hormone gene | |

LV-RTS-MMP1 components

| | | |
|---|---|---|
| IP | Inducible promoter | Drives expression of MMP1 |
| | | (therapeutic gene of interest) only in |
| | | the presence of activator ligand |
| RPL6-5′splice | 5′ untranslated region derived | 5′ regulatory element to enhance |
| unit | from the 60S ribosomal | transcription |
| | protein L5 5′ regulatory | |
| | element | |
| S100A6 | 3′ untranslated region derived | 3′ regulatory element to enhance |
| | from the human S100 | transcript stability |
| | calcium binding protein A6 | |
| | gene | |
| Const. P. | Constitutive promoter, e.g., | Drives expression of ecdysone |
| | Human elongation factor-1 | receptor-based gene switch |
| | alpha (EF-1 alpha) promoter | polypeptide components |
| VP16-RxR | Herpes simples virus (HSV) | The two fusion proteins form a |
| | protein 16 fusion with the | heterodimer in the presence of |
| | retinoid X receptor | activator ligand to form an active |
| Gal4 BD-EcR | Yeast Gal4 DNA biding | transcription factor that drives gene |
| | domain fusion with a | expression from the inducible |
| | modified ecdysone receptor | promoter (IP) |
| 2Xrbm3 IRES | Internal ribosome entry site | Allows for translation of two proteins |
| | derived from the RNA- | on a single mRNA transcript |
| | Binding Motif protein 3 | |
| HSV TK V3 3′ | 3′ untranslated region derived | Enhances stability of ecdysone |
| | from the HSV thymidine | receptor-based gene switch mRNA |
| | kinase gene | transcripts |

An embodiment of the invention includes the ability to control the expression of MMP or a collagen-degrading fragment thereof in the patient through the use of a ligand and gene switch system. The gene switch system may be any system that regulates gene expression of the therapeutic protein through the addition or removal of activator ligand. The components of the gene switch system include at least one inducible promoter, which is linked to the expression of a therapeutic protein operably linked thereto, a ligand-inducible transcription factor, a co-activation partner for the ligand-inducible transcription factor, and the activator ligand. The inducible promoter may be any promoter suitable for driving expression of the MMP gene.

Ligand-inducible transcription factors regulate gene expression by its interaction with a specified (small molecule) activator ligand and include any known transcription factors that will be controlled in the presence or absence of its corresponding activator ligand. By way of example, ligand-inducible transcription factors include members of the nuclear receptor superfamily activated by their respective ligands (e.g., glucocorticoid, estrogen, progestin, retinoid, ecdysone, vitamin D, and analogs and mimetics thereof) and the tetracycline-controlled transactivator (tTA) activated by tetracycline. In one aspect of the invention, the gene switch is an ecdysone receptor (EcR)-based gene switch, which comprises a heterodimeric protein complex comprising polypeptide sequences from at least two members of the nuclear receptor family, such as the ecdysone receptor (EcR) and ultraspiracle (USP) nuclear receptor protein families.

The activator ligand is the specific ligand that forms a complex with the ligand-inducible transcription factor, thereafter triggering the gene switch to stimulate expression of MMP. This ligand may include, for example, glucocorticoid, estrogen, progestin, retinoid, tetracycline, vitamin D, ecdysone, 20-hydroxyecdysone, ponasterone A, muristerone A, and the like, 9-cis-retinoic acid, synthetic analogs of retinoic acid, N,N′-diacylhydrazines, oxadiazolines, dibenzoylalkyl cyanohydrazines, N-alkyl-N,N′-diaroylhydrazines; N-acyl-N-alkylcarbonylhydrazines; N-aroyl-N-alkyl-N′-aroylhydrazines; amidoketones; 3,5-di-tert-butyl-4-hydroxy-N-isobutyl-benzamide, 8-O-acetylharpagide, oxysterols, 22(R) hydroxycholesterol, 24(S) hydroxycholesterol, 25-epoxycholesterol, T0901317, 5-alpha-6-alpha-epoxycholesterol-3-sulfate (ECHS), 7-ketocholesterol-3-sulfate, famesol, bile acids, 1,1-biphosphonate esters, juvenile hormone III, and the like. Examples of diacylhydrazine ligands useful in the present invention include RG-115819 (3,5-dimethyl-benzoic acid N-(1-ethyl-2,2-dimethyl-propyl)-N′-(2-methyl-3-methoxy-benzoyl)-hydrazide), RG-115932 (3,5-dimethyl-benzoic acid N-(1-tert-butyl-butyl)-N′-(2-ethyl-3-methoxy-benzoyl)-hydrazide), and RG-115830 (3,5-dimethyl-benzoic acid N-(1-tert-butyl-butyl)-N′-(2-ethyl-3-methoxy-benzoyl)-hydrazide). The activator ligand may optionally require other co-activation partners or ligands to form the complex needed to trigger the gene switch, as would be appreciated by one having skill in the art.

Examples of such systems include, without limitation, the systems described in U.S. Pat. Nos. 6,258,603, 7,045,315, U.S. Published Patent Application Nos. 2006/0014711, 2007/0161086, and International Published Application No. WO 01/70816. In one aspect of the invention, the gene switch is an EcR-based gene switch. Examples of such systems include, without limitation, the systems described in: PCT/US2001/009050 (WO 2001/070816); U.S. Pat. Nos. 7,091,038; 7,776,587; 7,807,417; 8,202,718; PCT/US2001/030608 (WO 2002/029075); U.S. Pat. Nos. 8,105,825; 8,168,426; PCT/US2002/005235 (WO 2002/066613); U.S. application Ser. No. 10/468,200 (U.S. Pub. No. 20120167239); PCT/US2002/005706 (WO 2002/066614); U.S. Pat. Nos. 7,531,326; 8,236,556; 8,598,409; PCT/US2002/005090 (WO 2002/066612); U.S. application Ser. No. 10/468,193 (U.S. Pub. No. 20060100416); PCT/US2002/005234 (WO 2003/027266); U.S. Pat. Nos. 7,601,508; 7,829,676; 7,919,269; 8,030,067; PCT/US2002/005708 (WO 2002/066615); U.S. application Ser. No. 10/468,192 (U.S. Pub. No. 20110212528); PCT/US2002/005026 (WO 2003/027289); U.S. Pat. Nos. 7,563,879; 8,021,878; 8,497,093; PCT/US2005/015089 (WO 2005/108617); U.S. Pat. Nos. 7,935,510; 8,076,454; PCT/US2008/011270 (WO 2009/045370); U.S. application Ser. No. 12/241,018 (U.S. Pub. No. 20090136465); PCT/US2008/011563 (WO 2009/048560); U.S. application Ser. No. 12/247,738 (U.S. Pub. No. 20090123441); PCT/US2009/005510 (WO 2010/042189); U.S. application Ser. No. 13/123,129 (U.S. Pub. No. 20110268766); PCT/US2011/029682 (WO 2011/119773); U.S. application Ser. No. 13/636,473 (U.S. Pub. No. 20130195800); PCT/US2012/027515 (WO 2012/122025); and, U.S. Pat. No. 9,402,919; each of which is incorporated by reference in its entirety.

In another aspect of the invention, a gene switch may be based on heterodimerization of FK506 binding protein (FKBP) with FKBP rapamycin associated protein (FRAP) and is regulated through rapamycin or its non-immunosuppressive analogs. Examples of such systems include, without limitation, the ARGENT transcriptional technology (ARIAD Pharmaceuticals, Cambridge, Mass.) and the systems described in U.S. Pat. Nos. 6,015,709, 6,117,680, 6,479,653, 6,187,757, and 6,649,595.

In another aspect of the invention, gene expression cassettes of the invention may incorporate a cumate switch system, which works through the CymR repressor that binds the cumate operator sequences with high affinity (e.g., SPARQ cumate switch (System Biosciences, Inc.)). The repression is alleviated through the addition of cumate, a non-toxic small molecule that binds to CymR. This system has a dynamic inducibility, can be finely tuned and is reversible and inducible.

In another aspect of the invention, gene expression cassettes of the invention may incorporate a riboswitch, which is a regulatory segment of a messenger RNA molecule that binds an effector, resulting in a change in production of the proteins encoded by the mRNA. An mRNA that contains a riboswitch is directly involved in regulating its own activity in response to the concentrations of its effector molecule. Effectors can be metabolites derived from purine/pyrimidine, amino acid, vitamin, or other small molecule co-factors. These effectors act as ligands for the riboswitch sensor, or aptamer. Breaker, RR. Mol Cell. (2011) 43(6):867-79.

In another aspect of the invention, gene expression cassettes of the invention may incorporate the biotin-based gene switch system, in which the bacterial repressor protein TetR is fused to streptavidin, which interacts with a synthetic biotinylation signal is fused to VP16 to activate gene expression. Biotinylation of the synthetic peptide is regulated by a bacterial biotin ligase BirA, thus enabling ligand responsiveness. Weber et al. (2007) Proc. Natl. Acad. Sci. U.S.A. 104, 2643-2648; Weber et al. (2009) Metabolic Engineering, 11 (2): 117-124.

Additional gene switch systems appropriate for use in the invention are known in the art, including but not limited to those described in Auslander and Fussenegger, Trends in Biotechnology (2012), 31(3):155-168, incorporated herein by reference.

The activator ligand is the specific ligand that forms a complex with the ligand-inducible transcription factor triggering the gene switch to stimulate expression of MMP. This ligand may include, for example, glucocorticoid, estrogen, progestin, retinoid, tetracycline, vitamin D, ecdysone, 20-hydroxyecdysone, ponasterone A, muristerone A, and the like, 9-cis-retinoic acid, synthetic analogs of retinoic acid, N,N′-diacylhydrazines such as those disclosed in U.S. Pat. Nos. 6,013,836; 5,117,057; 5,530,028; and 5,378,726 and U.S. Published Application Nos. 2005/0209283 and 2006/0020146; oxadiazolines as described in U.S. Published Application No. 2004/0171651; dibenzoylalkyl cyanohydrazines such as those disclosed in European Application No. 461,809; N-alkyl-N,N′-diaroylhydrazines such as those disclosed in U.S. Pat. No. 5,225,443; N-acyl-N-alkylcarbonylhydrazines such as those disclosed in European Application No. 234,994; N-aroyl-N-alkyl-N′-aroylhydrazines such as those described in U.S. Pat. No. 4,985,461; amidoketones such as those described in U.S. Published Application No. 2004/0049037; each of which is incorporated herein by reference and other similar materials including 3,5-di-tert-butyl-4-hydroxy-N-isobutyl-benzamide, 8-O-acetylharpagide, oxysterols, 22(R) hydroxycholesterol, 24(S) hydroxycholesterol, 25-epoxycholesterol, T0901317, 5-alpha-6-alpha-epoxycholesterol-3-sulfate (ECHS), 7-ketocholesterol-3-sulfate, framesol, bile acids, 1,1-biphosphonate esters, juvenile hormone III, and the like. Examples of diacylhydrazine ligands useful in the present invention include RG-115819 (3,5-Dimethyl-benzoic acid N-(1-ethyl-2,2-dimethyl-propyl)-N′-(2-methyl-3-methoxy-benzoyl)-hydrazide), RG-115932 (3,5-dimethyl-benzoic acid (R)—N-(1-tert-butyl-butyl)-N′-(2-ethyl-3-methoxy-benzoyl)-hydrazide), and RG-115830 (3,5-Dimethyl-benzoic acid N-(1-tert-butyl-butyl)-N′-(2-ethyl-3-methoxy-benzoyl)-hydrazide). See, e.g., U.S. patent application Ser. No. 12/155,111, and PCT Appl. No. PCT/US2008/006757, both of which are incorporated herein by reference in their entireties.

The activator ligand may optionally require other co-activation partners or ligands to form the complex needed to trigger the gene switch, as would be appreciated by one having skill in the art.

The inducible promoter of the present invention may be any promoter capable of driving expression of the therapeutic gene, the activation of which is triggered by the formation of an activation complex formed among the ligand-inducible transcription factor and the ligand activator (and optionally a co-activation partner). Promoters suitable for expression include, for example, CMV immediate early promoter, HSV thymidine kinase promoter, heat shock promoters, early and late SV40 promoters, LTRs from retroviruses, and metallothionein-I promoters. Other promoters known to control expression of genes in prokaryotic or eukaryotic cells or their viruses may also be used.

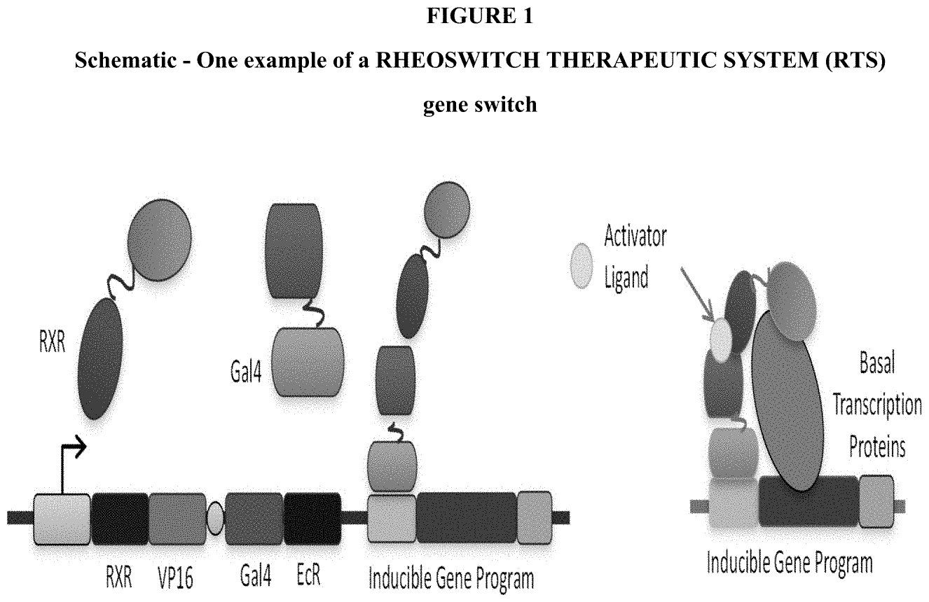

A preferred gene switch system for the present invention is an ecdysone receptor-based ligand-inducible gene switch, which allows regulated transgene expression under the control of the small molecule activator ligand, such as but not limited to veledimex. An ecdysone receptor-based gene switch contains three basic components: (1) an inducible promoter; (2) a ligand-inducible transcription factor and a co-activation partner; and (3) an activator ligand (AL) (such as, but not limited to, veledimex). In the absence of ligand, the gene switch protein complex provides an “off” signal and limits gene transcription. In contrast, in the presence of ligand, the complex provides a dose-dependent “on” signal for target gene (GOI) expression. A schematic of the control of ecdysone receptor-based regulated transgene expression is shown in FIG. 1.

Included in one example of an ecdysone receptor-based gene switch are two fusion proteins: Gal4/EcR and VP16/RXR. The coding sequences for both of these fusion proteins (Gal4-EcR and VP16-RXR) have been inserted in a replication-incompetent lentivirus vector and can be expressed in host cells following transduction and are described. Examples of ecdysone receptor-based gene switch fusions proteins are further described in PCT/US2002/005090, U.S. Pat. No. 8,715,959, PCT/US2008/011270, U.S. Pat. No. 9,402,919, and WO2009/045370, which are incorporated herein by reference.

Where an ecdysone receptor-based is used, a method according to the present invention may also include administration of a small molecular activator ligand, such as, but not limited to veledimex. Veledimex is a compound in the diacylhydrazine chemical class (DAH) of activator ligands. Veledimex (its USAN name) has a chemical name of: 3,5-dimethyl-benzoic acid (R)—N-(1-tert-butyl-butyl)-N′-(2-ethyl-3-methoxy-benzoyl)-hydrazide; or, N-[(1R)-1-(1,1-dimethylethyl)butyl]-N′-(2-ethyl-3-methoxybenzoyl)-3,5-dimethylbenzohydrazide; and, is also identified as INXN-1001 and RG-115932. Veledimex has the structural formula of Formula I:

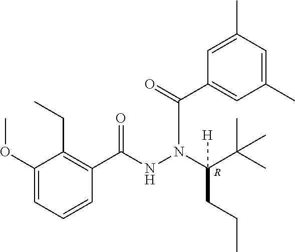

This ligand allows enhanced safety for control of the timing and level of transgene expression in gene and cell therapies. In the present invention, veledimex acts by binding to a Gal4-EcR ligand binding fusion protein which, in conjunction with a co-activation partner fusion protein (e.g., VP16/chimeric RxR/USP), activates mRNA expression of therapeutic gene transcription (MMP1), leading to synthesis and production of MMP1 protein (Palli et al., 2003) (Karzenowski et al., 2005).

In another aspect of the invention, cells are isolated or harvested from a patient suffering from sclerotic condition and transfected or transduced with a polynucleotide encoding the MMP protein or collagen-degrading fragment thereof. Thereafter, transfected or transduced cells are cultured ex vivo and subsequently administered to the patient from which they were originally harvested. If the polynucleotide expression cassette encoding the MMP1 protein includes a gene switch, the sclerotic condition patient may also be administered an activator ligand to activate the gene switch.

In another embodiment, transgene expression can be substantially confined to a desired site of action by the method of delivery, e.g., injecting within a sclerotic lesion. In combination with ligand activation this allows substantially confining expression of the effector within the diseased tissue where it has therapeutic action, thus minimizing systemic exposure and therefore reducing safety concerns.

Cells are extracted from the sclerotic condition patient via known methods and cultured to allow for their transfection or transduction with a polynucleotide encoding the MMP protein or a collagen-degrading fragment thereof or another protein with collagenase activity (e.g., enzymes that break the peptide bonds in collagen; is preferably done through the use of a viral vector. Any suitable viral vector for gene therapy delivery may be used. In the present invention, the viral vector is an adenoviral vector, an adeno-associated virus (AAV) or a lentiviral vector. Preferably, the viral vector is a lentiviral vector.

Lentiviral vectors used to construct the transducing vectors of the present invention are introduced via transfection or infection into a packaging cell line. The packaging cell line produces transducing vector particles that contain the vector genome. After co-transfection of the packaging vectors, transfer vector, and an envelope vector to the packaging cell line, the recombinant virus is recovered from the culture media and titered by standard methods used by those of skill in the art. Thus, the packaging constructs can be introduced into human cell lines by calcium phosphate transfection, lipofection or electroporation, optionally together with a dominant selectable marker, such as kanamycin, neomycin, DHFR, Glutamine synthetase or ADA, followed by selection in the presence of the appropriate drug and isolation of clones. The selectable marker gene can be linked physically to the packaging genes in the construct.

Stable cell lines wherein the packaging functions are configured to be expressed by a suitable packaging cell are known. For example, see U.S. Pat. No. 5,686,279; and Ory et al., (1996), which describe packaging cells. The packaging cells with a lentiviral vector incorporated in them form producer cells. Producer cells are thus cells or cell-lines that can produce or release packaged infectious viral particles carrying the therapeutic gene of interest. An example of a suitable lentiviral vector packaging cell lines includes 293 cells.

The copy number of the integrated transgene can be assessed using any known methods. For example, copy number may be determined through quantitative PCR, multiplex ligation-dependent probe amplification, fluorescent in situ hybridization (FISH), microarray-based copy number screening, and conventional karyotyping. The number of copies of the transgene integrated into each cell may be modulated by the virus dose given to the cells during production. The integrated transgene copy number per cell in the sclerotic condition harvested cells transduced with a MMP-containing vector is dose dependent.

In one embodiment, the number(s) of MMP or other collagen-degrading transgene in a cell is exactly, is about, is at least, or is not more than: 0.1, 0.2, 0.3, 0.4, 0.5, 0.6, 0.7, 0.8, 0.9, 1, 2, 3, 4 or 5 per cell.

In one embodiment, the number(s) of MMP or other collagen-degrading transgene integrated into a cell genome is exactly, is about, is at least, or is not more than: 0.1, 0.2, 0.3, 0.4, 0.5, 0.6, 0.7, 0.8, 0.9, 1, 2, 3, 4 or 5 per cell.

In certain embodiments, the number(s) of MMP or other collagen-degrading transgene in a cell is greater than 1 copies per cell.

In certain embodiments, the number(s) of MMP or other collagen-degrading transgene in a cell is less than 5 copies per cell.

In certain embodiments, the number(s) of MMP or other collagen-degrading transgene in a cell is greater than 1 and less than 5 copies per cell.

In certain embodiments, the number(s) of MMP or other collagen-degrading transgene in a cell is between about 1 and 5 copies per cell.

In certain embodiments, the number(s) of MMP or other collagen-degrading transgene in a cell is between about 2 and 5 copies per cell.

In certain embodiments, the number(s) of MMP or other collagen-degrading transgene in a cell is, between about 3 and 5 copies per cell.

In certain embodiments, the number(s) of MMP or other collagen-degrading transgene in a cell is between about 4 and 5 copies per cell.

In certain embodiments, the number(s) of MMP or other collagen-degrading transgene in a cell is about 5 copies per cell.

In certain embodiments, the number(s) of MMP or other collagen-degrading transgene integrated into a cell genome is greater than 1 copies per cell.

In certain embodiments, the number(s) of MMP or other collagen-degrading transgene integrated into a cell genome is less than 5 copies per cell.

In certain embodiments, the number(s) of MMP or other collagen-degrading transgene integrated into a cell genome is greater than 1 and less than 5 copies per cell.

In certain embodiments, the number(s) of MMP or other collagen-degrading transgene integrated into a cell genome is between about 1 and 5 copies per cell.

In certain embodiments, the number(s) of MMP or other collagen-degrading transgene integrated into a cell genome is between about 2 and 5 copies per cell.

In certain embodiments, the number(s) of MMP or other collagen-degrading transgene integrated into a cell genome is, between about 3 and 5 copies per cell.

In certain embodiments, the number(s) of MMP or other collagen-degrading transgene integrated into a cell genome is between about 4 and 5 copies per cell.

In certain embodiments, the number(s) of MMP or other collagen-degrading transgene integrated into a cell genome is about 5 copies per cell.

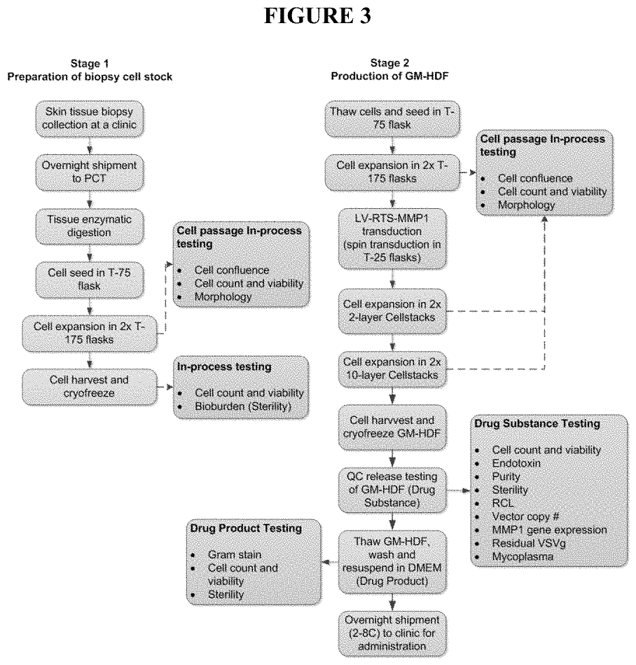

In another embodiment of the invention, LV-RTS-MMP vector may be used to transduce human dermal cells, such as fibroblasts, harvested from a biopsy of a sclerotic condition patient. These transduced human dermal cells (HDF) are cultured ex vivo and subsequently reintroduced into the same patient. In this manner, generation of the autologous cells genetically modified to carry the MMP gene may be done in stages. Stage 1 may encompasses biopsy, enzymatic digestion and initial cell expansion and biopsy cell stock cryofreeze. Stage 2 starts with thawing of frozen cell stock, cell expansion for LV-RTS-MMP transduction, additional cell expansion, cell harvest and cryofreeze to produce the transduced cells, which are subsequently administered back into the patient. Preferably, the cells harvested from the biopsy of a sclerotic condition patient are transduced with LV-RTS-MMP and these transduced cells are for administration back into the same sclerotic condition patient. In one embodiment, transduced cells are designated the “FCX-013” drug substance. FIG. 3 shows a high level process flow diagram within the scope of the invention for production of FCX-013 drug substance.

In accordance with the present invention, the intradermal administration of FCX-013 with the use of veledimex will locally increase MMP levels to degrade the excess collagen present in the sclerotic areas. Work in the field of mechanotransduction suggests that decreasing tissue stiffness may impact ongoing fibrosis by promoting an anti-fibrotic environment by increasing production of MMPs and anti-fibrotic agents such as prostaglandin in a feed-forward loop (Carver & Goldsmith, 2013).

Where a genetic switch requiring the use of veledimex as the ligand activator, the composition comprising veledimex may be formulated to any suitable concentration. The veledimex formulation may be encapsulated in various strengths, including, for example, 5 mg, 10 mg, 20 mg, 30 mg, 40 mg, 50 mg or 100 mg strength for oral administration. In some embodiments, use of a veledimex formulation is encapsulated at 5 mg, 10 mg, 20 mg, 30 mg, 40 mg, or 50 mg veledimex per capsule for oral administration. Veledimex may be administered daily for at least one week, two weeks, three weeks, one month, two months, three months, or six months. In some embodiments, veledimex is administered in an amount of 5 mg, 10 mg, 20 mg, or 30 mg once a day. In another preferred embodiment, the veledimex is administered in an amount of 40 mg once a day. Doses of veledimex may be administered twice daily, once daily, once every two days, once every three days or at other periodic intervals for at least 7 days, 10 days, 14 days, 21 days, 28 days, 30 days, 60 days, 90 days or more.

Veledimex acts by stabilizing heterodimerization between the two fusion proteins, forming an active transcription factor. This active transcription factor induces expression of a target transgene placed under the control of a ligand-inducible gene expression system (Kumar et al., 2004), (Lapenna et al., 2008), (Kumar et al., 2002), (Palli, 2003), (Shea & Tzertzinis, 2010), (Weis et al., 2009), (Katakam et al., 2006.)

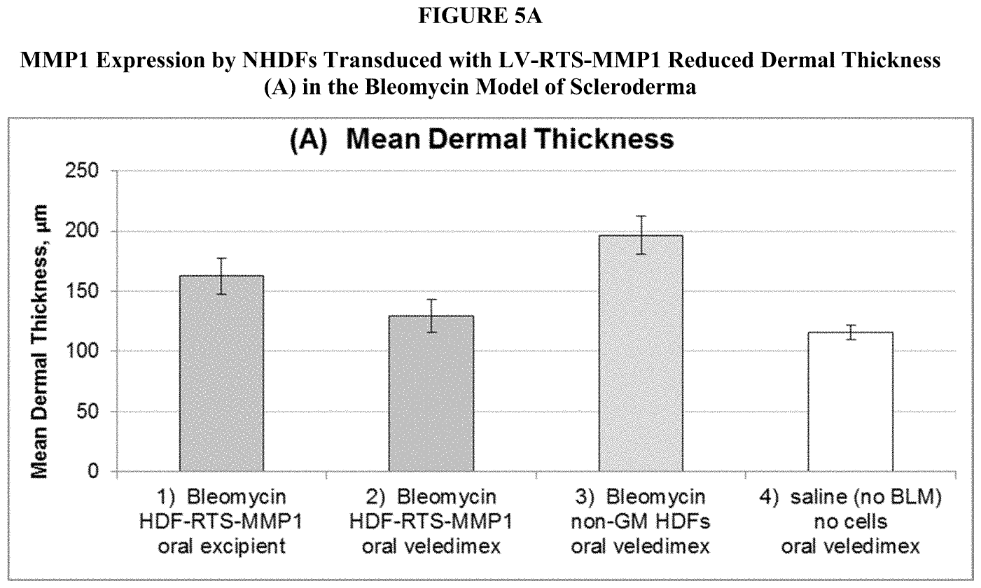

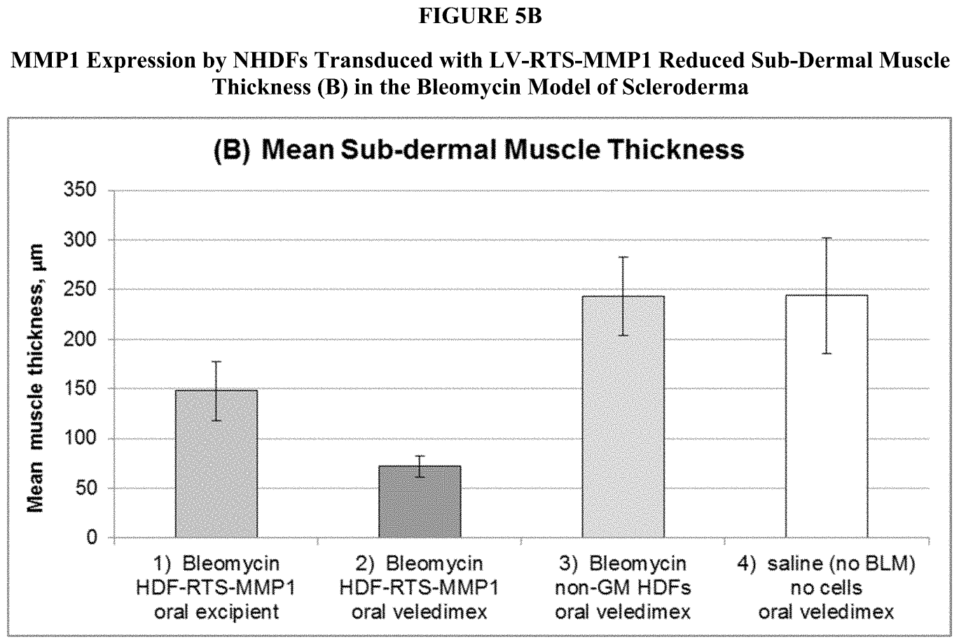

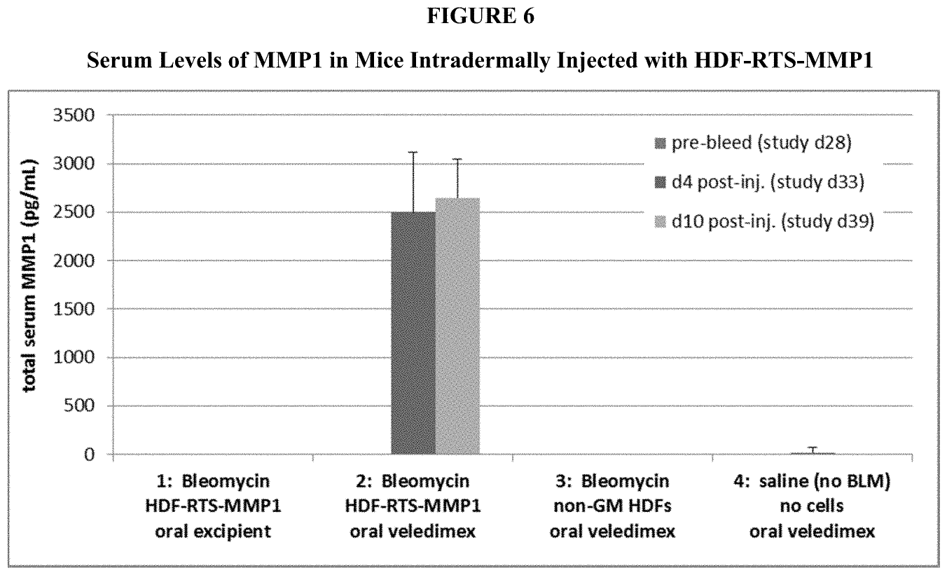

In certain embodiments, wherein a gene switch is incorporated into gene expression system, the presence or absence of activator ligand (such as but not limited to veledimex) can act to turn the expression of the MMP or a collagen-degrading fragment thereof on or off, respectively. For example, an in vitro study was conducted to determine the long-term kinetics for the expression of reference protein in mice where administration of veledimex was modified over time. The ON/OFF group durations for a protein operably linked to the reference gene switch in which veledimex is the ligand activator has been shown to be directly linked to the presence or absence of veledimex. In particular, Table 3 and FIGS. 5 and 6, for example, directly show that the presence or absence of veledimex in combination with the administration of autologous cells transduced with LV-RTS-MMP1 is directly correlated to the controlled expression of the MMP-1 protein operably linked to an ecdysone receptor-based gene switch. As those of ordinary skill in the art know and understand, “off” expression does not necessarily mean zero (0) detectable gene expression. Instead, “off” expression means that gene expression has been substantially reduced compared to “on” or the ligand activated gene expression state.

The present invention relates to the treatment and/or prevention of sclerotic conditions and diseases associated with excess collagen. Such conditions and diseases include morphea and localized scleroderma, including linear scleroderma, circumscribed morphea, generalized morphea, pansclerotic morphea, and mixed morphea as well as 1) Systemic Scleroderma (SSc) (specifically, scerodactyly and internal organ fibrosis); 2) skin fibrosis including: a) limited cutaneous SSc (ISSc) and b) diffuse cutaneous SSc (dSSc); 3) systemic sceroderma with interstitial lung disease (ILD) (SSc-ILD); 4) Edematous fibrosclerotic panniculopathy (cellulite); 5) Adhesive capsulitis (frozen shoulder syndrome); 6) Raynaud's phenomenon (RP); 7) psoriasis; 8) liver fibrosis (including nonalcoholic steatohepatitis); 9) kidney fibrosis (including, focal segmental glomerulosclerosis); 10) cardiac fibrosis; 11) rheumatoid arthritis; 12) Crohn's disease; 13) ulcerative colitis; 14) myelofibrosis; 15) systemic lupus erythematosus (SLE); 16) skeletal muscle fibrosis (following acute injury or due to chronic neurodegenerative muscular diseases); 17) congenital fibrosis of the extraocular muscle (CFEOM1, CFEOM2, CFEOM3 and Tukel syndrome); 18) chronic graft versus host disease; 19) hypertrophic scars; 20) idiopathic pulmonary fibrosis; 21) en coup de sabre; 22) dupuytren's contracture; 23) peyronie's disease; 24) hypertropic scars; 25) scleroderma associated hand dysfunction; 26) radiation fibrosis syndrome; and, 27) other sclerotic conditions.

Administration of the polynucleotide encoding MMP or collagen-degrading fragment thereof on is delivered by known methods suitable for delivering a gene directly to the skin of the patient. The polynucleotide may be delivered by injection, topically, or implantable devices. Each of the administrations could be preceded by a debriding of the affected tissue.

In one embodiment administration is by: 1) direct intralesion application (injection or topical); 2) embedding fibroblasts in a collagen matrix; 3) embedding fibroblasts in a hydrogel or mesh; 4) encapsulating fibroblasts in a polymer capsule; 5) intra-arterial injection of fibroblasts into the liver; and 6) general topical administration.

In one embodiment, the polynucleotide is delivered by injection. In another embodiment, the polynucleotide is delivered via viral vector in combination with the gene switch. In another embodiment, the polynucleotide is delivered to a harvested autologous cells, which is transduced with a viral vector encoding the polynucleotide encoding MMP, and the transduced cells is administered to the patient. In one embodiment, the vector comprising the MMP or collagen-degrading fragment thereof polynucleotide is administered a single time. In another embodiment, the vector is administered 1-2 times, 1-3 times, 1-4 times, or 1-5 times during the course of treatment. In another embodiment, autologous transduced cells comprising the MMP gene (or encoding a collagen-degrading fragment thereof) is administered 1-2 times, 1-3 times, 1-4 time, or 1-5 times during the course of treatment.

The polynucleotide encoding MMP or collagen-degrading fragment thereof is delivered in an amount sufficient to transduce cells to express the MMP protein or collagen-degrading fragment thereof. In one aspect of the invention, the INXN-2005 or LV-RTS-MMP vector is delivered into a cell harvested from the biopsy of a sclerotic conditions patient and the transduced cell is administered to the sclerotic conditions patient. The dosage of the vector comprising the MMP or collagen-degrading fragment thereof polynucleotide is sufficient to transduce cells to express a MMP gene product effective to induce a collagen-degrading effect as shown through the reduction of collagen I, II and/or III, or an anti-fibrotic effect. For example, the dosage of the vector comprising the polynucleotide encoding MMP or collagen-degrading or anti-fibrotic fragment thereof can be any suitable amount effective to reach the desired effect. Alternatively, the effective amount may be the amount needed to reduce, inhibit or prevent the fibrotic effect, ECM-accumulating, or collagen-forming effect, or the amount needed to reduce or inhibit sclerotic conditions. For example, a sclerotic conditions patient can be found to have reduced ECM accumulation by 10, 20, 30, 35, 40, 45, 50, or even 55% percent for patients treated with a vector comprising an ecdysone receptor-based gene expression system and comprising the MMP transgene, such as INXN-2005, in combination with veledimex relative to the sclerotic condition prior to treatment or relative to a patient treated with INXN-2005 without veledimex or a patient with no treatment. Preferably, the collagen-degrading effect results in reduced collagen formation or ECM accumulation by at least 50, or at least 55% percent for patients treated with a vector comprising an ecdysone receptor-based gene expression system and comprising the MMP transgene, such as INXN-2005, in combination with veledimex. Alternatively, the collagen-degrading effect produced from treatment of a sclerotic disease can be found to reduce collagen I, II and/or III or collagen formation by 10, 20, 30, 35, 40, 45, 50, or even 55% percent for patients treated with autologous cells comprising an ecdysone receptor-based gene expression system and the MMP-1 transgene, such as INXN-2005, in combination with veledimex relative to the sclerotic condition prior to treatment or relative to a patient treated with INXN-2005 without veledimex or a patient with no treatment. Preferably, the collagen-degrading effect results in reduced collagen formation by least 50, or at least 55% percent for patients treated with autologous cells comprising an ecdysone receptor-based gene expression system and comprising the MMP transgene, such as INXN-2005, in combination with veledimex. In some aspects, the treatment of sclerotic conditions or the decrease in collagen formation is correlated with a decrease in the concentration of collagen, I, collagen III, and/or TGFβ. In another aspect of the invention, inhibition of collagen formation is represented by an increase in IFN-gamma.

Where the polynucleotide encoding MMP or collagen-degrading fragment thereof is conditionally expressed through a gene switch system, the ligand activator is administered to the patient prior to, concurrently with, and/or subsequent to the administration of the polynucleotide encoding MMP or collagen-degrading fragment thereof. The ligand activator may be administered in any manner suitable to activate the gene switch, including by injection, topically, through implantable devices, or systemically, such as orally, intravenously, subcutaneous or intramuscular injection, parenteral injection, dermal delivery, or nasal delivery. Preferably, the ligand activator is administered orally. The ligand activator may be present in any suitable pharmaceutical carrier or may be delivered in a pharmaceutical composition designed for a sustained release system. The timing of the administration of the polynucleotide encoding MMP or collagen-degrading fragment thereof is preferably prior to the administration of the ligand activator. In some aspects of the invention, the ligand activator may be administered 1, 2, 3, 4, or 5 days after the injection of the polynucleotide encoding the MMP or collagen-degrading fragment thereof or after the injection of cells transfected or transduced with a polynucleotide encoding the MMP or collagen-degrading fragment thereof. The ligand activator may further be continually or intermittently administered daily, weekly or monthly to activate the expression of MMP or its collagen-degrading fragment. In one aspect of the invention, the ligand activator is administered daily for up to at least 20, at least 30, at least 40, at least 50, or at least 100 days after administration of the polynucleotide encoding MMP or collagen-degrading fragment thereof operably linked to a gene switch system or transduced cells comprising such polynucleotide. Where expression of the MMP or collagen-degrading fragment thereof is desired to be stopped, the ligand activator is no longer administered to the patient effectively turning the gene switch off. Preferably, the ligand activator is veledimex. It is conceivable that expression of the MMP or collagen-degrading fragment thereof may occur over the lifetime of a patient provided that the ligand activator is continually administered.

The ligand activator is delivered in an amount sufficient to activate the gene switch for the desired time period. For example, where an activator ligand (such as but not limited to veledimex) is administered daily, the dosages may be administered in 10-100 mg, 25-75 mg, 30-50 mg, or 40-50 mg strengths. One skilled in the art would be able to adjust the dosage of the ligand activator based on the delivery system and desired duration of effectiveness to activate the gene switch.

Pharmaceutical compositions of the present disclosure may include any suitable pharmaceutically acceptable carrier. Suitable carriers include, but are not limited to, water, dextrose, glycerol, saline, ethanol, and combinations thereof. The carrier can contain additional agents such as wetting or emulsifying agents, pH buffering agents, or adjuvants which enhance the effectiveness of the formulation. Topical carriers include liquid petroleum, isopropyl palmitate, polyethylene glycol, ethanol (95%), polyoxyethylene monolaurate (5%) in water, or sodium lauryl sulfate (5%) in water. Other materials such as antioxidants, humectants, viscosity stabilizers, and similar agents can be added as necessary.

The pharmaceutical compositions of this disclosure can include or be co-administered (concurrent, pre-treatment, or post-treatment) with: 1) botulinum toxin (e.g., Botox); 2) anti-IL6 biologic (e.g., tocilizumab); 3) anti-CD20 biologic (e.g., rituximab); 4) selective costimulation modulator (e.g., abatacept); 5) soluble guanylate cyclase stimulator; acts as vasodilator and anti-fibrotic agent (e.g., BAY63-2521); 6) betaglycan peptide; inhibits TGF-beta signaling (e.g., P144 cream); 7) CB2 receptor activator; inhibits immune responses; 8) anti-BLyS biologic; inhibits survival/differentiation of B cells) (e.g., belimumab); 9) PPAR activator, anti-fibrotic (e.g., IVA-337); 10) pyridone derivative (e.g., pirfenidone); 11) coagulation factor XIII; 12) clostridium collagenase; 13) thalidomide derivative—antiangiogenic and immunomodulator (e.g., CC-4047); 14) beta-catenin/CBP modulator; anti-fibrotic agent (e.g., C-82); 15) IL1-TRAP (e.g., IL1-TRAP); 16) oncostatin M mAb; anti-fibrotic/immunomodulator (e.g., GSK-2330811); 17) deuterium-containing pirfenidone (e.g., SD-560); 18) miR-29 analog (e.g., MRG-201); and 19) Adipose derived regenerative cells (e.g., ECCCS-50).

Pharmaceutical treatment kits or systems suitable for the treatment of sclerotic conditions are also within the scope of the present invention. A pharmaceutical treatment system or kit of the invention may include an injectable composition comprising the vector comprising the polynucleotide encoding MMP protein or collagen-degrading fragment thereof, which are linked to the gene switch system, and separately, the activator ligand used to activate the gene switch system.

### EXAMPLES

### Example 1

LV-RTS-MMP1 Lentivirus Construction and Characterization

The MMP1 gene was introduced to the cultured fibroblast cells using a recombinant Lentiviral Vector (LV), LV-RTS-MMP1. The LV is a replication incompetent, Vesicular Stomatitis Virus-G (VSV-G) pseudotyped, self-inactivating (3^{rd} generation) lentivirus (SIN-LV). LV-RTS-MMP1 has the nucleotide sequence corresponding to SEQ ID NO:1 and the amino acid sequence has the sequence of SEQ ID NO:3.

Source of the Human MMP1 Gene

The sequence for the MMP1 cDNA (SEQ ID NO:1) was derived from the consensus sequence of human pro-MMP1 with a replacement of the native MMP1 signal peptide with the signal peptide sequence of human Pigment epithelium-derived factor (PEDF) (SEQ ID NO:2) to provide more efficient secretion of MMP1 from fibroblasts. The cDNA (1410 bp) was generated and cloned into a standard expression plasmid for initial analyses. The MMP1 cDNA was then engineered to remove potential splice sites and cloned into the pFUGW SIN-LV backbone adjacent to an ecdysone receptor-based expression cassette, for MMP1 expression control using activator ligand, such as but not limited to, veledimex, to produce the LV-RTS-MMP1 vector. FIG. 2 provides a graphical representation of the LV-RTS-MMP1.

### Example 2: FCX-013 and Veledimex Background

### 2.1 In Vitro Studies

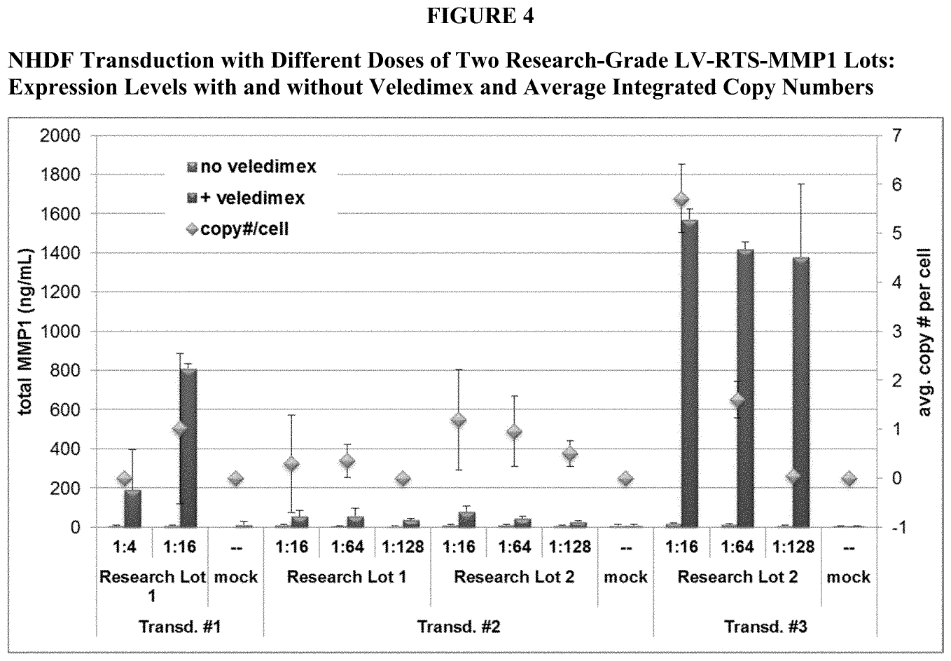

Veledimex induced expression of MMP1 was assessed in primary fibroblasts genetically modified by transduction with LV-RTS-MMP1. Primary normal human dermal fibroblasts (NHDFs) were transduced with varying dilutions of a research-grade LV-RTS-MMP1 stock. Following two passages post-transduction, transduced NHDFs (HDF-RTS-MMP1) were seeded into 24-well plates and treated with either 100 nM veledimex or 0.1% DMSO (vehicle). NHDFs not transduced with LV-RTS-MMP1 (“mock”) were also included in the studies. Cell supernatants were collected 72 h after the addition of veledimex or DMSO and analyzed for MMP1 expression levels by ELISA (R&D Systems DuoSet). Cells were also collected and analyzed for integrated LV-RTS-MMP1 copy numbers (average per cell) by qPCR using primers and probe specific to the LV-RTS-MMP1 construct. Results from three transduction studies are detailed below in Table 2 and graphically in FIG. 4. High levels of MMP1 expression were seen in the presence of veledimex. Un-induced levels were similar to mock-transduced levels with higher LV-RTS-MMP1 dilutions (lower MOTs). The average integrated copy numbers ranged with LV-RTS-MMP1 dose and copy numbers as high as 5.7 copies/cell were achieved.

TABLE 2
NHDF Transduction with Different Doses of Two Research-Grade LV-
RTS-MMP1 Lots: Expression Levels with and without Veledimex and
Average Integrated Copy Numbers

| Trans- | | | | | | |
|---|---|---|---|---|---|---|
| duction | | | | ng/mL MMP1 | | Avg |
| Study | LV-RTS- | Dilu- | | (±SD) | | Copy# |
| # | MMP1 | tion | MOT | no AL | +AL | (±SD) |
| 1 | Research | 1:4 | 144.6 | 6.8 ± 1.6 | 190 ± 204 | nd** |
| | Lot 1 | 1:16 | 36.1 | 9.2 ± 1.2 | 810 ± 24.7 | 1.01 ± 1.53 |
| | (3.47e7 | | | | | |
| | TU/mL) | | | | | |
| | mock | — | — | nd | 11.9 ± 16.8 | — |
| 2 | Research | 1:16 | 36.1 | 12.2 ± 1.7 | 56.1 ± 29.8 | 0.29 ± 0 |
| | Lot 1 | 1:64 | 9.0 | 6.1 ± 1.1 | 56.7 ± 37.8 | 0.35 ± .32 |
| | (3.47e7 | 1:128 | 4.5 | 5.1 ± 0.9 | 36.8 ± 4.9 | nd |
| | TU/mL) | | | | | |
| | Research | 1:16 | 37.4 | 10.9 ± 3.8 | 80.5 ± 25.0 | 1.19 ± 1.02 |
| | Lot 2 | 1:64 | 9.3 | 10.0 ± 2.6 | 43.9 ± 10.3 | 0.95 ± 0.71 |
| | (3.59e7 | 1:128 | 4.7 | 9.53 ± 2.0 | 27.9 ± 3.1 | 0.50 ± 0.25 |
| | TU/mL) | | | | | |
| | mock | — | — | 8.6 ± 5.0 | 9.0 ± 5.3 | — |
| 3 | Research | 1:16 | 37.4 | 19.9 ± 1.2 | 1571 ± 54 | 5.71 ± 0.69 |
| | Lot 2 | 1:64 | 9.3 | 14.7 ± 1.3 | 1421 ± 36 | 1.6 ± 0.36 |
| | (3.59e7 | 1:128 | 4.7 | 8.6 ± 2.4 | 1380 ± 371 | 0.05 ± 0.5 |
| | TU/mL) | | | | | |
| | mock | — | — | 6.1 ± 0.6 | 6.5 ± 0.7 | — |

TU = transducing units
MOT = multiplicity of transduction (TU/mL × volume ÷ dilution factor ÷ number of cells)
**nd = not determined

### 2.2 In Vivo Studies

A rodent model that totally recapitulates the disease phenotype of localized scleroderma/morphea is currently not available. Moreover, the rodent models that come close are immune-competent, precluding the assessment of gene-modified human cells. To address whether FCX-013 has the potential for efficacy, the bleomycin-induced scleroderma model, utilizing NOD/SCID mice, was selected to assess whether MMP1 expressed by GM-fibroblasts could reverse dermal fibrosis. HDF-RTS-MMP1 cells transduced with a 1:16 dose, with an average of 5.7 integrated copies/cell (Transduction #3 from Table 2 above) and mock-transduced cells (non-GM) were amplified further (to passage 6 post-transduction) to obtain adequate cell number for use in the in vivo study and then cryopreserved. A vial of each was thawed, cultured, and re-verified for inducible MMP1 expression by ELISA in the presence of veledimex (+AL) or 0.1% DMSO (no AL). Results are shown in Table 3 below.

TABLE 3
LV-RTS-MMP1 Transduced NHDFs Continue to
Express High Levels of MMP1 in the Presence
of Veledimex after Additional Passaging

| | No AL (ng/mL) | +AL (ng/mL) |
|---|---|---|
| Non-GM HDF (mock) | 5.7 | 3.8 |
| HDF-RTS-MMP1 | 15.6 | 1714 |

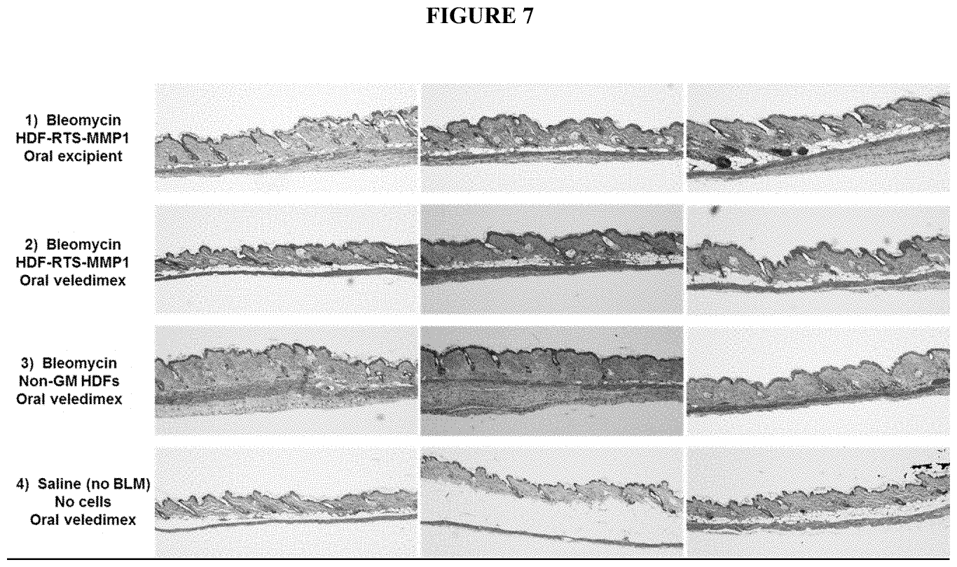

As detailed in the in vivo study design in Table 4 below, NOD/SCID mice received dermal injections of bleomycin (or saline; group 4) every other day for 4 weeks. The day following the last bleomycin treatment, mice were injected with either HDF-RTS-MMP1 cells (groups 1 and 2), non-modified cells (group 3), or no cells (no injection, group 4) into the same location as the bleomycin injections. Beginning the same day as cell injections, mice received veledimex (groups 2, 3, and 4) or excipient (capryo190/triacetin; group 1) by oral gavage for 10 consecutive days. On day 10 post-injection of cells, the injection sites were biopsied and preserved for histology. Histological sections were stained with H&E, imaged, and dermal thickness measured using ImageJ software (5 slides per mouse, 5 images per slide; 10× magnification, 1 pixel=0.8686 μm conversion). Randomly selected images for each mouse for each group are presented in FIG. 7. Serum was also collected from each mouse on study days 28, 33, & 39 (days −1 {pre-bleed}, 4, and 10 post-injection of fibroblasts) and assayed for circulating MMP1 (FIG. 6).

TABLE 4
In vivo Study Design for Examining Mechanism of Action of
HDF-RTS-MMP1 in the Bleomycin-Induced Scleroderma Model in NOD/SCID Mice

| | | | | Treatment | | | Treatment days | |
|---|---|---|---|---|---|---|---|---|
| | | | | | Treatment #2 | | (#2 & #3) | |
| | | | Treatment | Test | | Dose | HDF | AL |
| | | | #1 | Article | Test | (100 | dosing | dosing |
| Grp | N | Sex | Test Article | (50 μL) | Article | μL) | day | days |
| 1 | 8 | F | Intradermal, | Capryol90/ | HDF- | 5e5 | Day 29 | Days |
| | | | BLM, qod, | Triacetin, | RTS- | cells | | 29-39 |
| | | | d0-28 | oral | MMP1 | | | |
| 2 | 9 | F | Intradermal, | Veledimex, | HDF- | 5e5 | Day 29 | Days |
| | | | BLM, qod, | oral | RTS- | cells | | 29-39 |
| | | | d0-28 | | MMP1 | | | |
| 3 | 6 | F | Intradermal, | Veledimex, | HDFs | 5e5 | Day 29 | Days |
| | | | BLM, qod, | oral | (non- | cells | | 29-39 |
| | | | d0-28 | | GM) | | | |
| 4 | 7 | F | Intradermal, | Veledimex, | — | — | — | Days |
| | | | Saline, qod, | oral | | | | 29-39 |
| | | | d0-28 | | | | | |

Qod = every other day

The graph presented in FIG. 5 shows that treatment of bleomycin-induced lesions with intradermal injections of HDF-RTS-MMP1 cells reduces the thickness of the dermal layer (FIG. 5A) and the sub-dermal muscle layer (FIG. 5B). Moreover, induction of MMP1 expression by oral delivery of veledimex reduced the dermal thickness to levels similar to non-bleomycin (saline) treated skin (FIG. 5A) and further reduced the thickness of the sub-dermal muscle layer (FIG. 5B). The data suggests that even the low levels of MMP1 expression measured in vitro without veledimex activation, likely an artifact due to the high integrated LV-RTS-MMP1 copy number, had an impact on dermal and sub-dermal muscle thickness.

MMP1 was expressed in vivo by the cells used in this study at levels high enough to be detected systemically (FIG. 6). Although high levels of MMP1 are detected in the serum of vector plus veledimex treated animals, MMP1 is not detectable in serum of animals with vector without veledimex. This suggests that low levels of MMP1 may be sufficient to reduce dermal thickness.

### Example 3: Study in NOD/SCID Mice

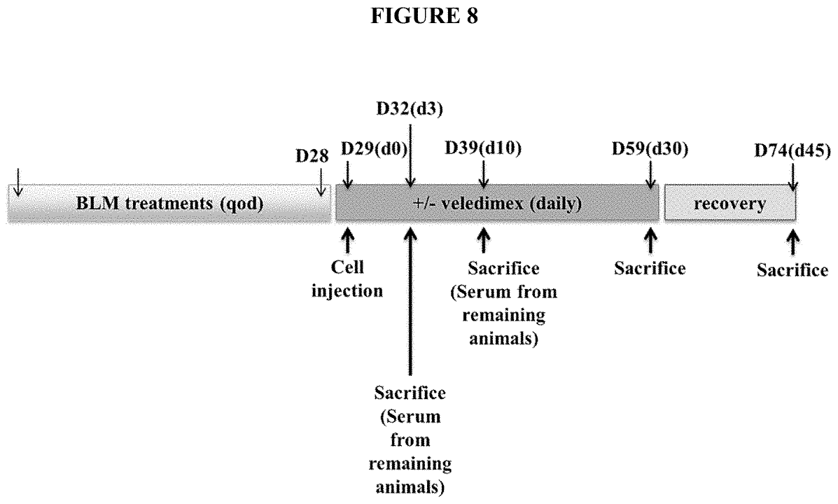

This example describes a study using a bleomycin-induced (BLM-induced) disease model in NOD/SCID mice. FIG. 8 shows an overview of treatment schedule.

Table provides a description of study groups.

Design of this study is premised on experimental data (data not shown) which showed:

In NOD/SCID mice administered FCX-013 plus veledimex, MMP1 expression (protein and mRNA) was maximal between 24 hrs and day 3 and undetectable by 28-days post-injection of FCX-013.

Protein expression was higher in BLM-treated animals compared to non-BLM-treated animals.

Systemic toxicity observed was attributable to bleomycin treatment.

No definitive toxicities were associated with 2×10⁶ FCX-013 cells

There was rapid decline of DNA copy numbers and MMP1 expression by 10-days post-injection of FCX-013

Copy number target should preferably be higher than 1 copy per cell to ensure sufficient MMP1 expression, and about 5 or less copies per cell for safety and efficacy in treating disorders such as scleroderma.

The objective of this example study is to evaluate toxicity, vector biodistribution, persistence of vector and MMP1 expression of intradermally injected FCX-013 cells and observing effects in normal and sclerotic skin of BLM-induced scleroderma model in NOD/SCID mice.

The study is conducted in a bleomycin-induced scleroderma model, utilizing NOD/SCID mice. NOD/SCID mice receive dermal injections of bleomycin or saline every other day (D or d) for 4 weeks (Day 1 (D1)) of study represents the initiation of treatment with BLM). The day following the last bleomycin treatment, mice are injected with either FCX-013 cells, non-modified cells, or no cells into the same location as the bleomycin injections. Beginning the same day as cell injections, mice receive veledimex or excipient by oral gavage for 30 consecutive days. In some groups there is a 14-day recovery period. Terminal assessments are conducted on 3, 10, 30 (and 45 in recovery groups) days post injection of Vehicle, FCX-013, or Non-GM-HDF cells. Serum is collected from each mouse on 3 and 10 days post injection of cells and assayed for circulating MMP1. Specifically, serum is collected at d3 (post injection of cells) from mice that are sacrificed at d10 and serum is collected at d10 (post injection of cells) from mice that are sacrificed at d30.

Study treatment groups are shown below.

TABLE 5
(a): Treatment Group

| | | Target | Target | | Target | | |
|---|---|---|---|---|---|---|---|
| | | Total | Dose | Total Dose | Dose | BLM Dose | Veledimex |
| | Test | Dose | per Site | Volume | Conc. | (μg) | Dose (μg/ |
| Group No. | Material | (cells) | (cells) | (μL)^{a} | (cells/mL) | (qod)^{b} | mouse)^{c} |
| 1 | Control | 0 | 0 | 200 | 0 | 0 | 0 |
| | (Vehicle) | | | | | | |
| 2 | Control | 0 | 0 | 200 | 0 | 10 | 1000 |
| | (Vehicle) | | | | | | |
| 3 | FCX-013 | 6 × 10⁶ | 3 × 10⁶ | 200 | 1 × 10⁷ | 0 | 1000 |
| 4 | FCX-013 | 6 × 10⁶ | 3 × 10⁶ | 200 | 1 × 10⁷ | 10 | 0 |
| 5 | FCX-013 | 6 × 10⁵ | 3 × 10⁵ | 200 | 1 × 10⁶ | 10 | 1000 |
| 6 | FCX-013 | 2 × 10⁶ | 1 × 10⁶ | 200 | 1 × 10⁷ | 10 | 1000 |
| 7 | FCX-013 | 6 × 10⁶ | 3 × 10⁶ | 200 | 1 × 10⁷ | 10 | 1000 |

(b): Treatment Group (tables continued from above)

| | | No. of Mice | | | | | | | |
|---|---|---|---|---|---|---|---|---|---|
| | | Day | | Day | | Day | | | |
| | | 32 | | 39 | | 59 | | Day 74^{d} | |
| Group | Test | (d3) | | (d10) | | (d30) | | (d45) | |
| No. | Material | M | F | M | F | M | F | M | F |
| 1 | Control | 1 | 1 | 1 | 1 | 1 | 1 | — | — |
| | (Vehicle) | 0 | 0 | 0 | 0 | 0 | 0 | | |
| 2 | Control | 1 | 1 | 1 | 1 | 1 | 1 | — | — |
| | (Vehicle) | 0 | 0 | 0 | 0 | 0 | 0 | | |
| 3 | FCX-013 | 1 | 1 | 1 | 1 | 1 | 1 | — | — |
| | | 0 | 0 | 0 | 0 | 0 | 0 | | |
| 4 | FCX-013 | 1 | 1 | 1 | 1 | 1 | 1 | — | — |
| | | 0 | 0 | 0 | 0 | 0 | 0 | | |
| 5 | FCX-013 | 1 | 1 | 1 | 1 | 1 | 1 | 5 | 5 |
| | | 0 | 0 | 0 | 0 | 0 | 0 | | |
| 6 | FCX-013 | 1 | 1 | 1 | 1 | 1 | 1 | 5 | 5 |
| | | 0 | 0 | 0 | 0 | 0 | 0 | | |
| 7 | FCX-013 | 1 | 1 | 1 | 1 | 1 | 1 | 5 | 5 |
| | | 0 | 0 | 0 | 0 | 0 | 0 | | |

__ = Notapplicable.
^{a}Administered once at 2 seperate dorsal sites (100 μL/site) on Day 29
^{b}Administered qod (every other day) by intradermal injections to two seperate dorsal sites for Groups 2-8 (100 μL/site) on Days 1 to 27
^{c}Daily oral dosing of 50 μL/dose on Days 29 to 39 or Days 29 to 59 (50 μL of 20 mg/mL solution is a dose of 1000 μg/mouse which equates to a dose of ~50 mg/kg for a 20 g mouse.
^{d}Recovery mice: veledimex administration is stopped after day 59

Intradermal route of administration, dose and regimen of the bleomycin reagent are selected to induce the dermal sclerosis model. The oral route of administration, doses, and regimen of the veledimex are selected because veledimex is given orally and the maximal dose encompasses a dose which may be given clinically. An intradermal route of administration of the test articles is selected because this may be a route of human administration.

In a previous study, treatment with LV-RTS-MMP1-modified HDFs harboring 5.7±0.69 average copies per cell, expressed 1571±54 ng/mL MMP1 protein in the presence of veledimex in vitro and resulted in a significant reduction in both dermal and sub-dermal muscle thickness in bleomycin-treated NOD/SCID mice. Moreover, in the absence of veledimex, there was an intermediate, yet significant reduction in dermal and sub-dermal muscle thickness. In vitro these 5.7 copy number cells expressed 19.9±1.2 ng/mL MMP1 protein in the absence of veledimex, which is similar to a level of MMP1 expressed in vitro in the presence of veledimex for the lower 0.83 copy number cells used in a different study. Based on the MMP1 expression levels from in vitro transduction studies, there may be a linear correlation between copy rate and MMP1 expression. Additionally, it was observed that ˜1 copy/cell of LV-RTS-MMP1 showed no discernible toxicity in mice, but also exhibited significantly lowered effectiveness in reducing both dermal thickness and dermal fibrosis. Accordingly, a copy number target of −5 copies/cell with groups having 3 different total number of cells (3×10⁵, 1×10⁶, 3×10⁶) are chosen as doses to be tested in the present study.

Procedures, Observations, and Measurements Viability Checks
Frequency: Are done twice daily, once in the morning and once in the afternoon, throughout the study.

### Clinical Observations

Frequency: At least once during the acclimation period, at least once before dose administration on day of dosing, and once daily thereafter.

### Post-Dose

Frequency: Clinical observations are recorded between 1 and 2 hours after dose administration and at the end of the day. Timing for post-dose observations may be extended. Clinical observations are recorded daily for remainder of post-dose period.

### Body Weights

Frequency: At least weekly during acclimation period. On the day of dose administration and at least twice weekly during the postdose period, including the day of scheduled euthanasia.

### Clinical Pathology

### Hematology

Frequency: On the days 3, 10, and 30 post injection of cells.

### Tissue Collection and Preservation for PCR

Representative samples of tissues identified in PCR Tissue Collection table are collected for cell specific quantitative polymerase chain reaction (qPCR) analysis using aseptic techniques. The abdomen is opened and blood is collected from the vena cava for hematology evaluation. A small section of tissues, including gross lesions/masses, when possible, is cut from the organs, and tissue section weights are recorded. Samples are snap-frozen on dry ice/alcohol bath immediately after weighing, placed in a cooler containing dry ice until placed in a freezer set to maintain (<−60° C.) until shipment for analysis.

Vector/mRNA qPCR

Injection sites and select list of tissues and major organs are evaluated by qPCR for vector and MMP1 specific mRNA.

### Example 4: Study in NOD/SCID Mice—Expression of MMP1 Via FCX-013

Biodistribution of FCX-013 cells and expression kinetics of MMP-1 were evaluated in nonobese diabetic/severe combined immunodeficiency (NOD/SCID) mice. FCX-013 plus veledimex biodistribution and expression were determined by evaluation of LV construct DNA and MMP-1 specific mRNA expression using qualified quantitative PCR assays. MMP-1 protein quantification was done using a commercially available kit. MMP-1 biodistribution and expression was evaluated following an intradermal injection of FCX-013 cells. Cells were injected intradermally into two sites (1×106 cells/site) on the dorsum of male and female immune-deficient NOD/SCID mice, followed by daily oral veledimex (48 mg/kg) for up to 28 days post FCX-013 injection.

Quantitative PCR methods for measuring integrated INXN-2005 (LV-RTS-MMP1) copy numbers (representing FCX-013 cells) and construct-specific MMP-1 mRNA expression levels were used to assess expression levels. The lower limit of detection and quantification for DNA copy numbers are 5 and 12.5 copies per 100 ng total DNA, respectively.

MMP-1 protein quantification was conducted a commercially available kit (Human MMP 3-Plex Ultra-Sensitive Kit from MesoScale Discovery (MSD; Rockville, Md., USA)); used for MMP-1 quantification in both serum samples and skin lysates (dynamic range of 11-100000 pg/mL) prepared according to manufacturer's recommendations.

Determination of veledimex in a dosing solution and in a dosing suspension was validated by high performance liquid chromatography (HPLC) with ultraviolet (UV) detection. Methods were validated for specificity, linearity, accuracy, precision, and stability during the assay period.

This study was to identify the onset, peak, and plateau of MMP-1 expression in mice treated with FCX-013 plus oral veledimex to inform optimal collection time points for the pivotal GLP toxicology study. Cells generated using the FCX-013 manufacturing method intended for use in a proposed clinical study were injected intradermally into two sites (1 million cells/site) on the dorsum of male and female immune-deficient NOD/SCID mice. The characteristics of these cells are indicated in Table 3. Mice then received either daily oral veledimex (48 mg/kg; Group 1) or excipient (Capryo190/Triacetin; Group 2) for up to 28 days post FCX-013 injection. At 6 h, 24 h, and 3, 7, 10, 14, 21 and 28 days a subset of mice in each group were euthanized for evaluations. Skin biopsies were collected and processed for either MMP-1 protein quantification by MSD ELISA or for INXN-2005 DNA and mRNA measurements by qPCR and RT-qPCR, respectively. Serum was also collected and tested for the presence of any systemic MMP-1 protein by MSD ELISA. Treatment groups and termination time points are summarized in Table 4.

TABLE 6
Characteristics of cells used in expression kinetics study

| | | In vitro MMP-1 expression levels | |
|---|---|---|---|
| | Integrated INXN- | Plus | No |
| | 2005 copy numbers¹ | veledimex | veledimex |
| FCX-013 | 6.98² | 162.9 ± 17.1 | 9.31± |
| Non-GMHDF | None detected | 1.79 ± 0.16 | 1.62± |

¹Cells were transduced with a pilot production lot of INXN-2005
²Integrated INXN-2005 (LV-RTS-MMP1) copy numbers of FCX-013

In skin biopsies, INXN-2005 copy numbers (representing FCX-013 cells) start to decline within 24 hours, but are still detectable at day 28 post-injection, with no significant differences between males and females and between veledimex- and excipient-treated animals (data not shown). MMP-1 mRNA expression is calculated as a ratio of the house-keeping gene-normalized Ct value of each sample and normalized to the average of Ct values for the 28 day post-injection time point without veledimex treatment. Results are expressed in FIG. 2 as fold-over the day 28 (without veledimex; AL) time point. Similar to DNA expression, mRNA expression peaked early (at 3 days post-injection of FCX-013) in mice orally treated with veledimex activator ligand, and was minimally detectable after 10 days post-injection.

Discussion:

Administration of intradermal FCX-013 and daily oral dosing with veledimex in NOD/SCID mice results in MMP-1 mRNA and protein expression which peaks between 24 hours and 3 days in the skin. MMP-1 expression levels were undetected after 10 days post-injection. The decline in expression levels over time was consistent with loss of INXN-2005 DNA copy numbers, representing FCX-013 cell numbers, which were still measurable at 28 days post-injection. In addition, nonclinical studies evaluating the control of expression of other target proteins by veledimex suggest that maximal expression of target protein can be achieved following oral administration of veledimex at doses that result in levels of veledimex in the tissue of approximately 250 ng/mL.

### Abbreviations and Acronyms

### Abbreviation or Acronym—Definition

BLM—Bleomycin
Cmax—The maximum observed concentration
CYP—Cytochrome
DMSO—Dimethylsulfoxide
ECM—Extracellular matrix
EcR—Ecdysone receptor
FCX-013—Genetically modified human dermal fibroblasts that express and secrete human matrix metalloproteinase 1 (MMP-1) under the control of a conditional (regulated) ecdysone receptor-based gene expression system
FXR—Famesoid X receptor
Gal4-EcR—Comprises DEF domains of a mutagenized EcR from the Spruce budworm (Choristoneura fumiferana) fused with the DNA binding domain of the yeast Gal4 transcription factor
GM—Genetically modified
GM-HDF—Genetically modified human dermal fibroblasts
INXN-1001—Veledimex Activator Ligand
INXN-2005—Lentiviral vector containing the MMP-1 gene construct (also known as LV-RTS-MMP-1)
IP—Intraperitoneal
IV—Intravenous
LV—Lentivirus
LV-RTS-MMP-1—Lentiviral vector containing the MMP-1 gene construct (also known as INXN-2005)
LTR—Long Terminal Repeat
MMP-1—Matrix metalloproteinase 1
MTD—Maximum tolerated dose
NA—Not available
NOD/SCID—Non-obese diabetic/severe combined immunodeficiency (NOD/SCID)
RAR—Retinoic acid receptor
RXR—Retinoid X receptor
SD—Sprague Dawley
USAN—United States Adopted Name
USP—Ultraspiracle
   VP16-RXR—The coding sequence consists of the EF domains of a chimeric (i.e., human and locust sequences) RXR fused with the transcription activation domain of the VP16 protein of HSV-1.

### REFERENCES

Asano, et al., “Involvement of alphavbeta5 integrin in the establishment of autocrine TGF-beta signaling in dermal fibroblasts derived from localized scleroderma,” J Invest Dermatol, vol. 126, no. 8, pp. 1761-9, 2006.
Bedair, et al., “Matrix metalloproteinase-1 therapy improves muscle healing,” J Appl Physiol, vol. 102, no. 6, pp. 2338-45, 2007.
Carver, et al., “Regulation of Tissue Fibrosis by the Biomechanical Environment,” BioMed Research International, vol. 2013, pp. 1-10, 2013.
Christen-Zaech, et al., “Pediatric morphea (localized scleroderma): review of 136 patients,” J Am Acad Dermatol, vol. 59, no. 3, pp. 385-96, 2008.
El-Mofty, et al., “Suggested mechanisms of action of UVA phototherapy in morphea: a molecular study,” Photodermatol Photoimmunol Photomed, vol. 20, no. 2, pp. 93-100, 2004.
Fett, et al., “Update on morphea: part I. Epidemiology, clinical presentation, and pathogenesis,” J Am Acad Dermatol, vol. 64, no. 2, pp. 217-28, 2011.
G. A. R. D. I. C. (GARD), “Localized Scleroderma,” 2012. [Online].
Giannandrea, et al., “Diverse functions of matrix metalloproteinases during fibrosis.”, Dis Model Mech. 2014 February; 7(2):193-203.
Grabbe, et al., “High-dose ultraviolet A1 (UVA1), but not UVA/UVB therapy, decreases IgE-binding cells in lesional skin of patients with atopic eczema,” J Invest Dermatol, vol. 107, no. 3, pp. 419-22, 1996.
Grundmann-Kollmann, et al., “PUVA-cream photochemotherapy for the treatment of localized scleroderma,” J Am Acad Dermatol, vol. 43, no. 4, pp. 675-8, 2000.
Gruss, et al., “Induction of interstitial collagenase (MMP-1) by UVA-1 phototherapy in morphea fibroblasts,” Lancet, vol. 350, no. 9087, pp. 1295-6, 1997.
Iimuro, et al., “Delivery of matrix metalloproteinase-1 attenuates established liver fibrosis in the rat,” Gastroenterology, vol. 124, no. 2, pp. 445-58, 2003.
Kaar, et al., “Matrix metalloproteinase-1 treatment of muscle fibrosis,” Acta Biomater, vol. 4, no. 5, pp. 1411-20, 2008.
Kelsey, et al., “The Localized Scleroderma Cutaneous Assessment Tool: responsiveness to change in a pediatric clinical population,” J Am Acad Dermatol, vol. 69, no. 2, pp. 214-20, 2013.
Kerscher, et al., “Treatment of localized scleroderma by UVA1 phototherapy,” The Lancet, vol. 346, no. 1166, pp. 91843-4, 1995.
Kroft, et al., “Period of remission after treatment with UVA-1 in sclerodermic skin diseases,” J Eur Acad Dermatol Venereol, vol. 22, no. 7, pp. 839-44, 2008.
Kumar, et al., “A single point mutation in ecdysone receptor leads to increased ligand specificity: Implications for gene switch applications,” Proc Natl Acad Sci USA, p. 14710-14715, 2002.
Kumar, et al., “Highly flexible ligand binding pocket of ecdysone receptor: a single amino acid change leads to discrimination between two groups of nonsteroidal ecdysone agonists,” J Biol Chem, vol. 279(26), pp. 27211-8, 2004.
Lapenna, et al., “Ecdysteroid ligand-receptor selectivity—exploring trends to design orthogonal gene switches,” FEBS J, pp. 5785-809, 2008.
Laxer, et al., “Localized Sclerodmera,” Curr Opin Rheumatol, vol. 18, no. 6, pp. 606-13, 2006.
Lipsker, et al., “Prospective evaluation of frequency of signs of systemic sclerosis in 76 patients with morphea,” Clin Exp Rheumatol, vol. 33, no. 4 Suppl 91, pp. S23-5, 2015.
Morita, et al., “Ultraviolet A1 (340-400 nm) phototherapy for scleroderma in systemic sclerosis,” J Am Acad Dermatol, vol. 43, no. 4, pp. 670-4, 2000.
Page-McCaw, et al., “Matrix metalloproteinases and the regulation of tissue remodelling.” Nat Rev Mol Cell Biol. 2007 March; 8(3):221-33.
Palli, et al., “Improved ecdysone receptor-based inducible gene regulation system,” European journal of biochemistry, pp. 1308-15, 2003.
Pardo, et al., “MMP-1: the elder of the family,” Int J Biochem Cell Biol, vol. 37, no. 2, pp. 283-8, 2005.
Pascual-Le, et al., “Effects of simulated solar radiation on type I and type III collagens, collagenase (MMP-1) and stromelysin (MMP-3) gene expression in human dermal fibroblasts cultured in collagen gels,” J Photochem Photobiol B, vol. 42, no. 3, pp. 226-32, 1998.
Peterson, et al., “The epidemiology of morphea (localized scleroderma) in Olmsted County 1960-1993,” J Rheumatol, pp. 73-80, 1997.
Piram, et al., “Short- and long-term outcome of linear morphoea in children,” Br J Dermatol, vol. 169, no. 6, pp. 1265-71, 2013.
Shea, et al., “Controlled expression of functional miR-122 with a ligand inducible expression system,” BMC biotechnology, vol. 10, p. 76, 2010.
Stege, et al., “High-dose UVA1 radiation therapy for localized scleroderma,” J Am Acad Dermatol, vol. 36, no. 6, pp. 938-44, 1997.
T. S. Foundation, “Localized Scleroderma,” 2015. [Online].
Takeda, et al., “Decreased collagenase expression in cultured systemic sclerosis fibroblasts,” J Invest Dermatol, vol. 103, no. 3, pp. 359-63, 1994.
Weiss, et al., “Inducible mutant huntingtin expression in HN10 cells reproduces Huntington's disease-like neuronal dysfunction. Molecular neurodegeneration,” Mol Neurodegener, pp. 1-12, 2009.
Yamamoto, “The bleomycin-induced scleroderma model: what have we learned for scleroderma pathogenesis?” Arch Dermatol Res. 2006 February; 297(8):333-44. Epub 2006 Jan. 10.
Yamamoto, et al., “Animal model of sclerotic skin. I: Local injections of bleomycin induce sclerotic skin mimicking scleroderma.” J Invest Dermatol. 1999 April; 112(4):456-62.
Yamamoto, et al., “Animal model of sclerotic skin. IV: induction of dermal sclerosis by bleomycin is T cell independent.” J Invest Dermatol. 2001 October; 117(4):999-1001.
Yamamoto, et al., “Animal model of systemic sclerosis.” J Dermatol. 2010 January; 37(1):26-41.
Zheng, et al., “UVA-induced ultrastructural changes in hairless mouse skin: a comparison to UVB-induced damage,” J Invest Dermatol, vol. 100, no. 2, pp. 194-9, 1993.

What is claimed:

1. A method of treating a sclerotic condition comprising administration of an expression vector, or cells comprising an expression vector, wherein said vector or cells comprise a polynucleotide encoding a fusion protein comprising a non-matrix metalloproteinase (non-MMP) signal peptide and a matrix metalloproteinase (MMP) polypeptide, or an enzymatically active collagen-degrading fragment thereof.

2. The method of claim 1, wherein the cells are first isolated from a patient suffering from scleroderma.

3. The method of claim 2, wherein the isolated cells are cultured ex vivo.

4. The method of claim 3, wherein the cells are fibroblasts.

5. The method of claim 1, wherein the polynucleotide encoding MMP or a collagen-degrading fragment thereof is further operably linked to a gene switch expression system.

6. The method of claim 5, wherein the gene expression switch system is activated to express MMP in the presence of an activator ligand and deactivated to reduce expression of MMP in the absence of the activator ligand.

7. The method of claim 1, wherein MMP is matrix metalloproteinase-1 (MMP-1).

8. The method of claim 7, wherein MMP-1 is human MMP-1.

9. The method of claim 1, wherein the expression vector is a viral vector.

10. The method of claim 8, wherein the viral vector is derived from a virus selected from lentivirus, adenovirus, and adeno-associated virus.

11. The method of claim 9, wherein the viral vector is a lentiviral vector.

12. The method of claim 6, wherein the activator ligand is veledimex.

13. The method of claim 1, wherein administration is by intradermal injection.

14. The method of claim 1, wherein veledimex is administered to the patient following injection of the transfected cells.

15. The method of claim 14, wherein the veledimex is delivered for at least five days after administration of cells.

16. The method of claim 1, wherein the sclerotic condition is scleroderma.

17. The method of claim 16, wherein the scleroderma is selected from linear scleroderma, circumscribed morphea, generalized morphea, pansclerotic morphea, and mixed morphea.

18. A lentiviral vector comprising a polynucleotide encoding a fusion protein comprising a non-matrix metalloproteinase (non-MMP) signal peptide and a matrix metalloproteinase (MMP) polypeptide, or an enzymatically active collagen-degrading fragment thereof operably linked to a gene switch system.

19. The lentiviral vector of claim 18, wherein the gene switch system comprises an inducible promoter operably linked to a ligand-inducible transcription factor.

20. The lentiviral vector of claim 19, wherein the gene switch system is activated in the presence of an activator ligand and deactivated in the absence of the activator ligand.

21. The lentiviral vector of claim 18, comprising the sequence of SEQ ID NO:1.

22. A lentiviral vector comprising the sequence of SEQ ID NO:1.

23. A pharmaceutical composition comprising a fibroblast obtained from a patient suffering from scleroderma transduced or transfected with a vector comprising the sequence of SEQ ID NO:1.

24. A cell transduced in vitro or ex vivo with the vector of claim 22.

25. An isolated genetically modified cell or population of genetically modified cells comprising a polynucleotide encoding a fusion protein comprising a non-matrix metalloproteinase (non-MMP) signal peptide and a matrix metalloproteinase (MMP) polypeptide, or an enzymatically active collagen-degrading fragment thereof operably linked to a gene switch system.

26. The genetically modified cell or population of genetically modified cells of claim 25, wherein the polynucleotide is present in the cell or population of cells, or integrated into the cell genome or population of cell genomes, at an average copy number of greater than 1 and less than 6 copies per cell.
